(19) United States
(12) Patent Application Publication
(10) Pub. No.: US 2006/0018885 A1
(43) Pub. Date: Jan. 26, 2006

(54) Methods for increasing HSC graft efficiency

(76) Inventor: Suzanne T. Ildstad, Prospect, KY (US)

Correspondence Address: HOGAN & HARTSON LLP, ONE TABOR CENTER, SUITE 1500, 1200 SEVENTEENTH ST, DENVER, CO, 80202, US

(21) Appl. No.: 11/004,046

(22) Filed: Dec. 3, 2004

Related U.S. Application Data
(63) Continuation-in-part of application No. 10/438,264, filed on May 14, 2003.
Application No. 10/438,264 is a continuation of application No. PCT/US01/45312, filed on Nov. 14, 2001.
(60) Provisional application No. 60/248,895, filed on Nov. 14, 2000.

(51) Int. Cl. A61K 48/00; C12N 5/08

(52) U.S. Cl. 424/93.7; 435/372

(57) ABSTRACT

This invention demonstrates that FC function via TNF-α to affect function of HSC. FC from TNF-α deficient mice are impaired in facilitating HSC engraftment in both the syngeneic and allogeneic models. Co-incubation of FC with HSC results in significant increase in TNF-α at the mRNA and protein level, and increase in transcript for Bcl-3 in HSC. Furthermore, neutralization of TNF-α results in the loss of FC ability to increase HSC clonogenicity and survival, as well as to upregulate Bcl-2 transcript in HSC, demonstrating a critical role for TNF-α in FC function. These results offer a mechanism of action for HSC regulation by accessory cells in the bone marrow and confirm the advantage of their co-transplantation with HSC to improve graft efficiency.

### CROSS-REFERENCE TO RELATED APPLICATIONS

This application is a Continuation-in-part application of U.S. patent application Ser. No. 10/438,264, filed May 14, 2003, which is a Continuation application under 35 USC § 1.111 (a) of International Application No. PCT/US01/45312, filed Nov. 14, 2001, which claims priority to U.S. Provisional Application Ser. No. 60/248,895, filed Nov. 14, 2000, the disclosures of which are incorporated herein by reference.

### CONTRACTUAL ORIGIN OF THE INVENTION

This research was supported in part by the National Institutes of Health, grant DK43901-07. The government has certain rights in the invention.

### BACKGROUND OF THE INVENTION

1. Field of the Invention

The present invention relates to the identification and use of facilitating cells that are critical for engraftment of purified hematopoietic stem cells (HSC). More specifically, this invention relates to the role of TNF-α production by FC in protecting HSC from undergoing apoptosis and in increasing engraftment efficiency and clonogenicity.

2. Description of the State of Art

The transfer of living cells, tissues, or organs from a donor to a recipient, with the intention of maintaining the functional integrity of the transplanted material in the recipient, defines transplantation. Transplants are categorized by site and genetic relationship between the donor and recipient. An autograft is the transfer of one's own tissue from one location to another; a syngeneic graft (isograft) is a graft between identical twins; an allogeneic graft (homograft) is a graft between genetically dissimilar members of the same species; and a xenogeneic graft (heterograft) is a transplant between members of different species.

After transplantation, HSC migrate from the peripheral blood and home to hematopoietic organs. This process is relatively inefficient, and during this process, approximately 70% of the transplanted purified HSC are lost due to early differentiation or cell death (Plett P A, et al., Blood 2003;102:2285-2291). Methods to enhance the efficiency of HSC engraftment after transplantation and increase efficiency of HSC self-renewal during ex vivo expansion could have a major impact on HSC transplantation, especially when HSC numbers are limiting.

A major goal in solid organ transplantation is the permanent engraftment of the donor organ without a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and if stopped, graft rejection usually results. However, a major problem in using nonspecific immunosuppressive agents is that they function by suppressing all aspects of the immune response, thereby greatly increasing a recipient's susceptibility to opportunistic infections, rate of malignancy, and end-organ toxicity (Dunn, D. L., Crit. Care Clin., 6:955 (1990)). Although immunosuppression prevents acute rejection, chronic rejection remains the primary cause of late graft loss (Nagano, H., et al., Am. J Med. Sci., 313:305-309 (1997)).

For every organ, there is a fixed rate of graft loss per annum. The five-year graft survival for kidney transplants is 74% (Terasaki, P.I., et al., UCLA Tissue Typing Laboratory (1992)). Only 69% of pancreatic grafts, 68% of cardiac transplants and 43% of pulmonary transplants function 5 years after transplantation (Opelz, G., Transplant Proc., 31:31S-33S (1999)).

The only known clinical condition in which complete systemic donor-specific transplantation tolerance occurs is when chimerism is created through bone marrow transplantation (Qin, et al., J. Exp. Med., 169:779 (1989); Sykes, et al., Immunol. Today, 9:23-27 (1988); and Sharabi, et al., J. Exp. Med., 169:493-502 (1989)). This has been achieved in neonatal and adult animal models as well as in humans by total lymphoid or body irradiation of a recipient followed by bone marrow transplantation with donor cells. The success rate of allogenic bone marrow transplantation is, in large part, dependent on the ability to closely match the “major histocompatability complex” (MHC) of the donor cells with that of the recipient cells to minimize the antigenic differences between the donor and the recipient, thereby reducing the frequency of host-versus-graft responses and “graft-versus-host disease” (GVHD). In fact, MHC matching is essential—only a one or two antigen mismatch is acceptable because GVHD is very severe in cases of greater disparities.

The MHC is a cluster of closely linked genetic loci encoding three different classes (class I, class II, and class III) of glycoproteins expressed on the surface of both donor and host cells that are the major targets of transplantation rejection immune responses. The MHC is divided into a series of regions or subregions, and each region contains multiple loci. An MHC is present in all vertebrates, and the mouse MHC (commonly referred to as H-2 complex) and the human MHC (commonly referred to as the Human Leukocyte Antigen or HLA) are the best characterized.

The development of safe methods to achieve mixed allogeneic chimerism to induce donor-specific tolerance across MHC barriers remains a major goal. Two barriers associated with bone marrow transplantation (BMT) have limited its application to clinical transplantation: (1) graft-versus-host disease (GVHD) and (2) failure of engraftment. T-cell depletion (TCD) of donor marrow can eliminate GVHD but is associated with a significant increase in graft failure. Consequently, it was hypothesized that T-cells are required for durable engraftment of allogeneic hematopoietic stem cells (HSC). Although highly purified HSC engraft readily in syngeneic and MHC-congenic recipients, they do not engraft as readily in MHC-disparate recipients. The addition of CD8⁺/TCR graft facilitating cells (FC) overcomes this limitation in mouse. In the rat, depletion of CD8⁺, CD3⁺ or CD5⁺ cells from the donor marrow is associated with a significant increase in failure of engraftment.

The role of MHC was first identified for its effects on tumor or skin transplantation and immune responsiveness. Different loci of the MHC encode two general types of antigens which are class I and class II antigens. In the mouse, the MHC consists of 8 genetic loci: Class I is comprised of K and D, class II is comprised of I-A and /or I-E. The class II molecules are each heterodimers, comprised of I-Aα and I-Aβ and/or I-Eα and I-Eβ. The major function of the MHC molecule is immune recognition by the binding of peptides and the interaction with T-cells, usually via the αβT-cell receptor. It was shown that the MHC molecules influence graft rejection mediated by T-cells (Curr. Opin. Immunol., 3:715 (1991), as well as by NK cells (Annu. Rev. Immunol., 10:189 (1992); J. Exp. Med., 168:1469 (1988); Science, 246:666 (1989)). The induction of donor-specific tolerance by HSC chimerism overcomes the requirement for chronic immunosuppression (Ildstad, S. T., et al., Nature, 307:168-170, (1984), Sykes, M., et al., Immunology Today, 9:23-27 (1998), Spitzer, T. R., et al., Transplantation, 68:480-484, (1999)). Moreover, bone marrow chimerism also prevents chronic rejection (Colson, Y., et al., Transplantation, 60:971-980 (1995); and Gammie, J. S., et al., Circulation (1998)). The association between chimerism and tolerance has been demonstrated in numerous animal models including rodents (Ildstad, S. T., et al. Nature, 307:168-170, (1984); and Billingham, R. E., et al., Nature, 172:606 (1953)), large animals, primates and humans (Knobler, H. Y., et al., Transplantation, 40:223-225 (1985); Sayegh, M. H., et al., Annals of Internal Medicine, 114:954-955 (1991)).

T-cells can be divided into two populations: αβ-TCR⁺ T-cells and γδ-TCR⁺ T-cells. αβ-T-cell receptor (TCR)⁺ T-cells are the predominant circulating population and can be subdivided into cells expressing CD4⁺ or CD8⁺ antigens. γδ-TCR⁺ T-cells represent approximately 2% of peripheral T-cells and are predominantly CD3⁺ CD4⁻/CD8⁻. The role of αβ-TCR⁺ T-cells in the pathophysiology of acute GVHD is supported by a number of studies. The role of γδ-TCR⁺ T-cells as effector cells for GVHD has been debated. Data from recently developed transgenic murine models indicate that a clonal population of γδ-TCR⁺ T-cells are capable of inducing acute GVHD, as well as mediating graft rejection. Blocking the ability of the TCR to bind to the host MHC through the use of peptides that target the MHC has led to reduction in GVHD. Elucidating the participation of αβ- and γδ-TCR⁺ subsets in GVHD is a necessary step in the goal of removing the T-cells responsible for GVHD, and on evaluating the influence of the cellular subsets on engraftment.

Highly purified hematopoietic stem cells (HSC) engraft readily in syngeneic and MHC congenic recipients while engraftment is significantly impaired in MHC-disparate allogeneic recipients. The addition of CD8⁺ graft facilitating cells (FC) restores engraftment-potential of highly purified HSC in allogeneic recipients in vivo (Sharkas, Martin, Weissman, Ildstad JEM; Kaufman, et al. Blood, 84:2436-2446 (1994)). The precise phenotype source and biological role of CD8⁺ FC has remained controversial (Martin, Immunity). As few as 10,000 CD8⁺/TCR⁺/CD3ε bone marrow-derived FC have been demonstrated to enable durable engraftment of HSC in fully ablated (950 cGy TBI) mice (Kaufman, Blood; Colson, Nat. MED). CD8⁺/TCR⁺ lymph node-derived FC are essential to engraftment of marrow in MHC disparate recipients conditioned with 800 cGy TBI (Martin, JEM). In mice conditioned with 800 cGy TBI, CD8⁺/TCR⁻ bone marrow-derived FC facilitated engraftment, but CD8^{total} (TCR⁺ plus TCR⁻) cells combined mediated the most potent engraftment-enhancing biologic effect (Weissman, Immunity). Because the majority of CD8⁺/TCR⁻ cells in marrow are CD3ε⁻, it was concluded that the biologic activity resided in this cellular fraction rather than the more infrequent CD3ε⁺ population (Weissman, Immunity).

Two populations of CD8⁺ cells in bone marrow have been described to facilitate engraftment of highly purified hematopoietic stem cells (HSC) in MHC-disparate allogeneic recipients. CD8⁺/TCR⁻ facilitating cells (FC) facilitate durable engraftment of HSC without causing GVHD, while CD8⁺/TCR⁻FC plus CD8⁺/TCR⁺ cells may also facilitate. CD8⁺/TCR⁺ cells alone are not sufficient to support long-term graft survival. Without FC, HSC prolong survival, but do not promote sustained engraftment.

HSC self-renewal is a complex process, and its regulatory mechanisms are poorly defined. Most models for study have relied upon in vitro cell culture and growth factor manipulations. The proliferation and differentiation of primitive hematopoietic progenitor cells are regulated by direct interaction of those cells with bone marrow stroma and by the stimulatory and inhibitory effects of cytokines present in the bone marrow microenvironment (Ogawa, 1993).

TNF-α is a pleitropic cytokine that plays a pivotal role in regulating HSC survival directly and via upregulation of other cytokine receptors (Rebel V I, et al., J. Experimental Medicine 1999; 190:1493-1504; Shieh J H, et al., J. Immunol. 1989; 143:2534-2539; Elbaz O, et al., J. Clin. Inves.t 1991;87:838-841; Jacobsen F W, et al., J. Immunol. 1995; 154:3732-3741; Durig J, et al., Blood 1998; 92:3073-3081). TNF-α has been shown to be a bifunctional regulatory of hematopoietic stem and progenitor cells, depending upon the growth factors used as supplements, as well as the maturation stage of the cell (Zhang, 1995). Most notably, TNF-α inhibits the growth of primitive HSC, preventing entry into s-phase from G₀/G₁ during stimulation by growth factors (Zhang, 1995). Further, signaling via the p55 subunit of the TNF receptor is essential for regulating hematopoiesis at the skin cell level (Rebel V I, et al., J. Experimental Medicine 1999; 190:1493-1504), and TNF-α protects primitive HSC from the stimulatory effects of ambient hematopoietic stimulators by TGF-β (Snoeck, 1996).

TNF-α affects cells through two distinct receptors (TNFR1: p55 TNFR and TNFR2: p75 TNFR). It can be expressed in a membrane-anchored form or in a secreted soluble form (Kriegler, 1988; Kinkhabwala, 1990. TNF-α inhibits the proliferation of primitive HSC and stimulates the proliferation of more committed hematopoietic progenitors, depending on the maturation of the cells as well as the presence of other cytokines in the media. Notably, HSC from p55 TNFR^{-/-} mice are impaired in self-renewal, implicating the p55 receptor as the inhibitory receptor for TNF-α function on HSC (Rebel V I, et al., J. Experimental Medicine 1999, 190:1493-1504; Jacobsen F W, et al., J. Immunol. 1995; 154:3732-3741). Until now, however, the cells within the hematopoietic microenvironment that mediate this regulatory effect via TNF-α have not been defined.

Bone marrow transplantation (BMT) has the potential to treat a number of genetic disorders, including hemoglobinopathies (sickle cell disease, thalassemia), soluble enzyme deficiencies, and autoimmune disorders. The morbidity and mortality associated with transplantation of unmodified marrow has prevented the widespread application of this approach. Conventional T-cell depletion prevents graft versus host disease but is associated with an unacceptably high rate of graft failure. A better understanding of the biology of engraftment of HSC will allow approaches to graft engineering to provide efficient engraftment and avoid the risks associated with BMT. Therefore, there remains a need for efficient engraftment procedures.

### SUMMARY OF THE INVENTION

Accordingly, certain embodiments of this invention provide methods for conditioning a recipient for bone marrow transplantation which minimize or eliminate the need for nonspecific immunosuppressive agents and/or lethal irradiation. More specifically, present teachings demonstrate that CD8⁺/TCR⁺/CD3ε⁺facilitating cells (FC) are critical to durable HSC engraftment, while CD8⁺/TCR⁻/CD3⁺ T-cells are only supplemental. Moreover, the present teachings demonstrated that TCR βδ KO mice produce FC, while CD3ε transgenic (TG) mice do not, suggesting a lymphoid-derived non T-cell lineage for CD8⁺/TCR⁻ FC.

This invention further identifies which cells in the host recipient microenvironment influence alloresistance to engraftment.

In various embodiments, the methods of this invention comprise introducing CD8⁺/TCR⁻ facilitating cells and purified hematopoietic stem cells into a recipient lacking T-cells.

In certain embodiments of the invention, methods are provided for depleting or eliminating those cells in the host environment which influence alloresistance to engraftment, thereby conditioning the recipient for engraftment.

This invention further demonstrates that TNF-a production by FC may protect HSC from undergoing apoptosis by up-regulating anti-apoptotic transcripts such as Bcl-3. More specifically, the present invention further demonstrates that FC require TNF-α to facilitate HSC engraftment in allogeneic and in syngeneic recipients in vivo, as well as in their role of enhancing HSC clonogenicity in vitro.

The present invention further demonstrates that TNF-α is involved in the ability of FC to upregulate Bcl-3 transcription into HSC.

Accordingly, certain embodiments of the present invention provide a method of increasing HSC engraftment survival, comprising co-incubating prior to engraftment a pharmaceutical composition that stimulates TNF-α expression and a cellular composition comprising human hematopoietic stem cells to form a mixture, wherein said stem cells are depleted of graft-versus-host-disease-producing cells having a phenotype of αβTCR+ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and administering said mixture to said mammal.

This invention further provides a method of increasing HSC engraftment survival in a mammal, comprising: co-incubating prior to engraftment a pharmaceutical composition that stimulates upregulation of Bcl-3 by said HSC and a cellular composition comprising human hematopoietic stem cells to form a mixture, wherein said stem cells are depleted of graft-versus-host-disease-producing cells having a phenotype of αβTCR+ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and administering said mixture to said mammal.

This invention further provides a method of increasing HSC engraftment survival in a mammal, comprising:administering to said mammal a cellular composition comprising human hematopoietic stem cells, wherein said stem cells are depleted of graft-versus-host-disease-producing cells having a phenotype of αβTCR+ with the retention of mammalian hematopoietic facilitatory cells having a phenotype of CD8+/TCR+, CD8+/TCR−, which hematopoietic facilitatory cells are capable of facilitating engraftment of bone marrow cells; and concurrently therewith or subsequent thereto, administering to said mammal a pharmaceutical composition that stimulates TNF-α expression.

This invention further provides a method of increasing HSC engraftment survival in a mammal, comprising administering to said mammal a cellular composition comprising human hematopoietic stem cells, wherein said stem cells are depleted of graft-versus-host-disease-producing cells having a phenotype of αβTCR⁺ with the retention of mammalian hematopoietic facilitatory cells having a phenotype of CD8+/TCR+, CD8+/TCR−, which hematopoietic facilitatory cells are capable of facilitating engraftment of bone marrow cells; and concurrently therewith or subsequent thereto, administering to said mammal a pharmaceutical composition that increases upregulation of Bcl-3 by said HSC.

This invention further provides a cellular composition comprising: a) mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and b) a pharmaceutical composition that provides an increased amount of TNF-α. In certain embodiment, the pharmaceutical composition comprises TNF-α. In certain other embodiments, the pharmaceutical composition comprises an agent that stimulates expression of TNF-α from said facilitatory cells.

This invention further provides a cellular composition comprising: a) mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and b) a pharmaceutical composition that provides an increased amount of Bcl-3.

This invention further provides a method of partially or completely reconstituting a mammal's lymphohematopoietic system comprising(a) administering to the mammal a cellular composition comprising mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and (b) stimulating the expression of TNF-α from said facilitatory cells and/or increasing the ability of said facilitatory cells to upregulate Bcl-3 in said hematopoietic stem cells. In certain embodiments, said stimulation comprises introducing to said facilitatory cells a pharmaceutical composition that increases TNF-α expression prior to administration to said mammal. In certain embodiments, said increase in upregulation is effected by introducing to said facilitatory cells a pharmaceutical composition that increases said upregulation of Bcl-3 prior to said administration. In certain embodiments, the method comprises administering to said mammal a pharmaceutical composition that increases TNF-α expression subsequent to said administration of said cellular composition. In certain embodiments, the method comprises administering to said mammal a pharmaceutical composition that increases said upregulation of Bcl-3 subsequent to said administration of said cellular composition.

This invention further provides a method of inducing tissue or organ regeneration in a mammal comprising: (a) administering to the mammal a cellular composition comprising mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and (b) stimulating the expression of TNF-α from said facilitatory cells and/or increasing the ability of said facilitatory cells to upregulate Bcl-3 in said hematopoietic stem cells.

In certain embodiments, said stimulation comprises introducing to said facilitatory cells a pharmaceutical composition that increases TNF-α expression prior to administration to said mammal. In certain embodiments, said increase in upregulation is effected by introducing to said facilitatory cells a pharmaceutical composition that increases said upregulation of Bcl-3 prior to said administration. In certain embodiments, the method comprises administering to said mammal a pharmaceutical composition that increases TNF-α expression subsequent to said administration of said cellular composition. In certain embodiments, the method comprises administering to said mammal a pharmaceutical composition that increases said upregulation of Bcl-3 subsequent to said administration of said cellular composition.

This invention further provides a method of protecting mammalian hematopoietic stem cells from apoptosis, comprising: depleting said stem cells of graft-versus-host-disease-producing cells having a phenotype of αβTCR⁺ while retaining mammalian hematopoietic facilitatory cells having a phenotype of CD8⁺/TCR⁺, CD8⁺/TCR⁻, which hematopoletic facilitatory cells are capable of facilitating engraftment of bone marrow cells; and contacting said depleted stem cells with a pharmaceutical composition that increases the amount of TNF-α.

This invention further provides a cellular composition comprising human hematopoietic stem cells (HSC), which are depleted of graft-versus-host-disease-producing cells having a phenotype of αβ-TCR⁺ and which have retained mammalian hematopoietic facilitatory cells having a phenotype of CD8⁺/TCR³¹ , CD8⁺/TCR⁺, which facilitatory cells have an increased expression of TNF-α and/or increase ability to upregulate Bcl-3 transcription in said HSC compared to wild-type FC, and which FC are capable of facilitating engraftment of bone marrow cells.

The present invention further provides methods for conditioning a recipient for bone marrow transplantation using the compositions of this invention. More specifically, certain embodiment of this invention provide methods for conditioning a recipient for bone marrow transplantation comprising introducing CD8⁺/TCR⁻ facilitating cells and purified hematopoietic stem cells into a recipient lacking T-cells, and stimulating the expression of TNF-α in FC and/or increasing the uptake of Bcl-3 by HSC. In certain embodiments, expression of TNF-α and/or uptake in Bcl-3 is increased by introducing to the FC an agonist that stimulates the expression of TNF-α and/or uptake in Bcl-3.

In certain embodiments, the stimulation occurs prior to the introduction of the HSC to the recipient. In certain other embodiments, the stimulation occurs subsequent to introduction of the HSC to the recipient.

Methods of increasing expression of TNF-α or uptake in Bcl-3 according to various embodiment of this invention include introducing to the FC an agonist that increases expression of TNF-α or uptake in Bcl-3.

Methods are also provided herein for treating a variety of diseases and disorders with minimized morbidity.

Additional novel features of this invention shall be set forth in part in the description and examples that follow, and in part will become apparent to those skilled in the art upon examination of the following or may be learned by the practice of the invention. The advantages of the invention may be realized and attained by means of the instrumentalities and in combinations particularly pointed out in the appended claims.

### BRIEF DESCRIPTION OF THE DRAWINGS

The accompanying drawings, which are incorporated in and form a part of the specifications, illustrate the preferred embodiments of the present invention, and together with the description serve to explain the principles of the invention. The skilled artisan will understand that the drawings, described below, are for illustration purposes only. The figures are not intended to limit the scope of the invention in any way.

In the Drawings:

FIG. 1 illustrates T-cell depletion of rat bone marrow.

FIGS. 2A, 2B, and 2C illustrate the detection of facilitating cells.

FIG. 3 illustrates the analysis of CD8⁺/TCR⁻ FC for expression of CD11a and CD11c.

FIG. 4 is a table illustrating the assessment of BVHD after bone marrow transplantation.

FIGS. 5A-E illustrate histologic assessments of GVHD.

FIG. 6 illustrates the survival of heterotopic cardiac allografts in mixed allogeneic chimeras (ACI→WF).

FIG. 7A illustrates the synthesis of TNF-α by FC after contact with HSC.

FIG. 7B illustrates the presence of intracellular TNF-α after 18 hours co-culture with HSC.

FIG. 7C illustrates the increase of TNF-α on the surface of FC after 18 hours co-culture with HSC.

FIG. 7D illustrates the production of cytokine by FC when co-cultured with HSC.

FIG. 8A illustrates the number of CFC per 1000 HSC in supernatants from culture of FC+HSC for 14 days in methylcellulose containing growth factors.

FIG. 8B illustrates the number of CFC per 1000 HSC in supernatants from culture of FC+HSC for 14 days in methylcellulose containing growth factors.

FIG. 8C illustrates the percent live HSC in supernatants from culture of FC+HSC for 14 days in methylcellulose containing growth factors.

FIG. 8D illustrates the percent of apoptotic HSC in supernatants from culture of FC+HSC for 14 days in methylcellulose containing growth factors.

FIG. 9A illustrates the survival of allogeneic recipients (B10.BR) conditioned with 950 cGy TBI and transplanted with 10,000 B6 HSC (▴) in presence or in absence of 30,000 FC from B6 (♦) or FC from TNF-α^{-/-} mice (□) compared to mice conditioned only (O).

FIG. 9B illustrates FC function in syngeneic recipients limiting HSC numbers. B6 mice were conditioned with 950 cGy TBI and transplanted with 1000 B6 HSC (●); 500 B6 HSC (▴); 500 B6 HSC and 30,000 B6 FC (□) or 500 B6 HSC plus 30,000 TNF-α^{-/-} FC (▪). Survival was calculated by the Kaplan-Meier method (P<0.01).

FIG. 10A illustrates the amount of CFC per 1,000 HSC after culture at 1:3 ratio with FC for 14 days in methylcellulose containing growth factor in the presence of FC or TNF-α^{-/-} FC or TNF-α^{-/-} FC pretreated with TNF-α (10 ng/mL for 1 hour).

FIG. 10B illustrates the amount of CFC per 1,000 HSC after culture at 1:3 ratio with FC for 14 days in methylcellulose containing growth factor after 18 hour preincubation.

FIG. 10C illustrates the amount of CFC per 1,000 HSC after culture at 1:3 ratio with FC for 14 days in methylcellulose containing growth factor in the presence of FC or TNF-α (10 ng/mL).

FIG. 10D illustrates the percent live HSC after incubation with FC or TNF-α^{-/-} FC.

FIG. 10E illustrates the percent apoptotic HSC after incubation with FC or TNF-α^{-/-} FC.

FIG. 11 illustrates the number of CFC per 1,000 HSC after preincubating HSC for 18 hours alone or at 1:3 ratio (HSC:FC) in the presence of FC or in the presence of FC pretreated with anti-TNF-α neutralizing antibody for 1 hour.

FIG. 12A illustrates the percent live HSC after preincubation for 40 hours alone or at 1:3 ratio (HSC:FC) in the presence of FC, or in the presence of FC pretreated with anti-TNF-α neutralizing antibody for 1 hour.

FIG. 12B illustrates the fold of difference of anti-apoptotic regulatory proteins mRNA after preincubation for 16 hours alone or at 1:3 ratio (HSC:FC) in the presence of FC, or in the presence of FC pretreated with anti-TNF-α neutralizing antibody for 1 hour.

FIG. 12C illustrates the fold of difference of Bcl-3 mRNA as evaluated after preincubation for 16 hours alone or at 1:3 ratio (HSC:FC) in the presence of FC, or in the presence of FC pretreated with anti-TNF-α neutralizing antibody for 1 hour.

FIG. 13A is a survival curve for B10.BR mice transplanted with allogeneic B6 HSC. B10.BR mice were conditioned with 950 cGy TBI and transplanted with 10,000 B6 HSC (□) in the presence or absence of 30,000 FC from B6 (▪) or with 10,000 HSC from TNFR^{-/-} mice (Δ) in presence of in absence of 30,000 WT FC (▴).

FIG. 13B illustrates the analysis of donor repopulation at 3 months in peripheral blood of recipients B 10.BR.

FIG. 13C illustrates donor repopulation of white blood cells at 3 months in recipients of TNFR^{-/-HSC}+FC compared to WT HSC+FC.

### DETAILED DESCRIPTION OF THE PREFERRED EMBODIMENT

A. Methods of Using CD8⁺/TCR⁻ FC for the Engraftment of Purified HSC

One aspect of the present invention is based on the hypothesis that CD8⁺/TCR⁻ FC are critical to HSC survival and self-renewal, while CD8⁺/TCR⁺ conventional T-cells are supplemental and do not promote long-term, durable engraftment. Further, donors lacking TCR⁻β/δ may still produce facilitating cells (FC). And, that depletion of αβ- and γδ-TCR⁺ T-cells will not affect the engraftment-potential of the rat bone marrow cells, since their depletion should leave the FC population intact.

In certain embodiments, a bone marrow transplant (BMT) was engineered in which the αβ- and γδ-TCR⁺ T-cells were depleted from donor marrow. The role of each cell population in engraftment and graft-versus-host disease (GVHD) was subsequently evaluated. Depletion of both αβ- and γδ-TCR⁺ T-cells from donor marrow allowed durable engraftment, but completely avoided GVHD. The resulting chimeric animals exhibited stable mixed allogeneic chimerism and donor-specific tolerance to cardiac grafts for one year. These data are consistent with the hypothesis that FC, although CD3⁺, are not “conventional” T-cells, because they do not express T-cell receptor (TCR). The present invention indicates that αβ- or γδ-TCR⁺ T-cells are sufficient to cause GVHD, and that the presence of either αβ- or γδ-TCR⁺ T-cells in the donor marrow inoculum affects the level of donor chimerism. These data confirm that neither αβ- nor γδ-TCR⁺ T-cells are required for durable HSC engraftment in MHC-disparate recipients, but that both contribute to GVHD as well as to influence the level of donor chimerism.

GVHD currently limits the clinical application of BMT for the induction of donor specific tolerance. Strategies to T-cell deplete the bone marrow of GVHD-producing cells prevent GVHD, but are associated with a significant increase in failure of engraftment. The rat is a superior model to study GVHD and TCD graft failure because it is more prone to GVHD as well as failure of engraftment compared to the mouse. Depletion of T-cells from the rat marrow using anti-CD5, anti-CD8, or anti-CD3 mAb decreases the incidence of GVHD but also results in increased occurrence of graft failure after allogeneic bone marrow transplant. A cell population in mouse bone marrow (CD8⁺/CD3⁺/CD5⁺/TCR⁻), separate from the HSC, facilitates engraftment of purified allogeneic HSC without causing GVHD. Because the FC shares some cell surface molecules with T-cells, it is not known whether the T-cell depletion-related graft failure is due to the depletion of facilitating cell populations or conventional T-cell populations. Recent studies suggest that the CD8⁺/TCR⁺ and CD8⁺/TCW subpopulations of marrow facilitate the engraftment of allogeneic HSC, but that the CD8⁺/TCR⁻ cells are the most potent effector cells and have the added advantage that they do not cause GVHD. Moreover, a facilitating role for CD8⁺ lymph node lymphocytes and γδT-cells has also been reported. In continuing studies in the mouse, purified FC allow physiologic numbers of HSC to engraft in allogeneic recipients, while purified T-cells do not. However, purified T-cells enhance engraftment in partially conditioned mouse recipients if FC are present.

In the present invention, it has been determined whether and how αβ- and γδ-TCR⁺ T-cells contribute to engraftment of HSC. It was hypothesized that in the previous studies the TCD strategy was removing FC as well as T-cells, resulting in graft failure, and that removal of T-cells (αβ- and γδ-TCR⁺ T-cells) with sparing of FC would not result in impaired engraftment. Virtually all recipients of marrow depleted of either αβ- or γδ-TCR⁺ T-cells engrafted. Similarly, all the recipients transplanted with donor marrow aggressively depleted of αβ- and γδ-TCR⁺ T-cells engrafted and exhibited stable mixed HSC chimerism. These data therefore demonstrate that depletion of αβ- and γδ-TCR⁺ T-cells allows engraftment of allogeneic HSC.

It is important to note that the CD3⁺/CD8⁺/TCR⁻ FC cell population remained in the donor cell inoculum after αβ- and γδ-TCR⁺ T-cell depletion. The ontogeny of FC and lineage derivation have not yet been defined. The FC population is separate from the conventional T-cell population when analyzed by flow cytometry in that CD8 and CD3 expression are less intense than that for CD8⁺ T-cells. Moreover, the FC population is predominantly CD11c positive, suggesting a possible dendritic cell ontogeny. Taken together, these data therefore indirectly support the existence of a facilitating cell population, separate from conventional T-cells, in rat bone marrow. Because there is no strategy currently available to purify rat HSC, the inventor was unable to sort only FC plus HSC and co-administer them in purified form to ablated rat recipients.

Although TCD did not influence engraftment, the percentage of donor chimerism was significantly influenced by the composition of the marrow inoculum. The role of γδ-TCR⁺ T-cells in influencing engraftment has been debated. Recipients of marrow depleted of both αβ plus γδ-TCR⁺ T-cells repopulated with significantly lower levels of mixed chimerism compared to those administered marrow containing αβ-TCR⁺ T-cells (46.3±32.8% and 92.3±9.2%, respectively; p<0.05). Moreover, recipients of marrow containing γδ-TCR⁺ T-cells also exhibited higher levels of donor chimerism. These data suggest that while conventional T-cells are not required for engraftment of the HSC, they do influence the level of chimerism established. These data resolve the apparent dichotomy between the report of facilitating cells and others in which lymph node CD8⁺ T-cells were demonstrated to enhance the level of chimerism, since FC were present in the marrow used in these studies. Moreover, while αβ- or γδ-TCR⁺ T-cells are not required for durable engraftment, they do significantly influence the level of chimerism.

Also evaluated was the role of T-cell subsets in mediating GVHD and influencing engraftment potential. It has been debated whether γδ-TCR⁺ T-cells can mediate GVHD. One study showed that the γδ-TCR⁺ T-cell does not play a role in GVHD in mice, while another showed that cells co-expressing γδ-TCR⁺ and natural killer (NK)1.1⁺ play a role in the pathogenesis of acute GVHD. However, the mouse is an inferior model for these studies because it is much more resistant to GVHD. The rat is more prone to GVHD and is therefore a superior model. Although the depletion of γδ-TCR⁺ T-cells alone did not significantly affect the development of GVHD, the depletion of γδ-TCR⁺ T-cells in addition to αβ-TCR⁺ completely avoids GVHD. None of these animals exhibited clinical signs of GVHD, while only minimal signs of GVHD (grade 1) were detected histologically. These data clearly demonstrate that although the αβ-TCR⁺ T-cells play a dominant role, γδ-TCR⁺ T-cells also contribute in an independent fashion to GVHD. It has been previously demonstrated that depletion of αβ-TCR⁺ T-cells from donor marrow decreased the occurrence of GVHD while preserving engraftment in rats. The results reported here are consistent with those results, indicating that αβ-TCR⁺ T-cells are important in mediating GVHD in rats. Although all the recipients reconstituted with marrow depleted of αβ-TCR⁺ T-cells but containing γδ-TCR⁺ T-cells exhibited clinical or histological signs of GVHD, the severity of the disease was decreased compared with recipients of marrow containing αβ-TCR⁺ T-cells. These data confirm that, while αβ-TCR⁺ T-cells mediate GVHD in the rat, γδ-TCR⁺ T-cells are also capable of inducing GVHD independent of αβ-TCR⁺ T-cells.

The selective depletion of marrow of either αβ- or γδ-TCR⁺ T-cell subsets allowed the evaluation of whether the specific cell types resulted in a differential occurrence of GVHD. Recipients of marrow depleted of only γδ-TCR⁺ T-cells developed moderate to severe GVHD relatively early after BMT (30 days post-transplantation), primarily affecting the skin and tongue. Depletion of only αβ-TCR⁺ T-cells resulted in milder GVHD affecting primarily in the liver and small intestine at 150 and 220 days post-BMT. However, when both αβ- and γδ TCR⁺ T-cells were depleted, severe GVHD was prevented. These data suggest that αβ- and γδ-TCR⁺ T-cell subsets target different tissues and mediate their affect at different times. αβ-TCR⁺ T-cells result in GVHD histologically by destruction of skin, tongue early post-BMT; γδ-TCR⁺ T-cells have the capability of causing GVHD target in liver and small intestine late post-BMT.

The induction of tolerance has the potential to overcome the two major problems that currently limit organ transplantation: chronic rejection and the complications associated with immunosuppressive therapy. Mixed allogeneic chimerism induces donor-specific transplantation tolerance to solid organ grafts. It has been debated whether donor T-cells must be present in the marrow inoculum for tolerance to be achieved. Such T-cells were hypothesized to “balance” the recipient T-cells. It is hypothesized that mixed chimerism induces deletional tolerance and that donor T-cells are not required for tolerance to be induced. The mixed chimeras generated using marrow depleted of both αβ- and γδ-TCR⁺ T-cells exhibit donor-specific tolerance to solid organ grafts. Mixed chimeras accept donor-specific cardiac grafts (MST>375 days) without evidence of chronic rejection while third-party cardiac grafts are rejected as rapidly as untreated control rats. These data therefore confirm that mature donor T-cells are not required to induce tolerance through mixed HSC chimerism. The results of the present invention culminate in the hypothesis that the engraftment of the donor pluripotent HSC in the form of mixed chimerism allows deletional tolerance to occur as newly produced host- and donor-derived lymphocytes are produced. The presence of donor-derived dendritic cells in the thymus of mixed chimeras provides a potent deleting ligand for any donor-reactive T-cells of host or donor origin, resulting in a robust form of tolerance.

In summary, the present invention demonstrates that αβ- and γδ-TCR⁺ T-cells affect the level of donor chimerism but not engraftment, since depletion of αβ- and γδ-TCR⁺ T-cells from the donor bone marrow retains engraftment potential yet avoids GVHD, suggesting that an FC population is present functionally as well as phenotypically in rat bone marrow. Moreover, both αβ- and γδ-TCR⁺ T-cells mediate GVHD. However, αβ-TCR⁺ T-cells mediate more severe GVHD with a more rapid onset than the GVHD mediated by γδ-TCR⁺ T-cells. Strategies to engineer a BMT to remove GVHD-producing cells but retain facilitating cells may allow the clinical application of BMT to induce tolerance to solid organ and cellular grafts to become a reality.

Thus, certain embodiments of the present invention relate to a composition comprising two cell populations of CD8⁺ cells, that is, CD8⁺/TCR⁻ facilitating cells (FC) which are critical to hematopoietic stem cells (HSC) survival and self-renewal, and CD8⁺/TCR⁺ cells which enhance the level of donor engraftment but do not promote long-term, durable engraftment.

Generally, purified or partially purified FC facilitate engraftment of stem cells which are MHC-specific to the FC so as to provide superior survival of the chimeric immune system. The stem cells and FC preferably come from a common donor or genetically identical donors. However, if the donor is of a species or a strain of a species which possesses a universal facilitatory cell, the stem cells need not be MHC-specific to the facilitatory cell. By purifying the FC separately, either by positive selection, negative selection, or a combination of positive and negative selection, and then administering them to the recipient along with MHC-specific stem cells and any desired additional donor bone marrow components, GVHD causing T-cells may be removed without fear of failure of engraftment. As a result, mixed or completely or fully allogeneic or xenogeneic repopulation can be achieved.

Typically methods of establishing an allogeneic or xenogeneic chimeric immune system comprises substantially destroying the immune system of the recipient. This may be accomplished by techniques well known to those skilled in the art. These techniques result in the substantially full ablation of the bone marrow-stem cells of the recipient. However, there may be some resistant recipient stem cells which survive and continue to produce specific immune cells. These techniques include, for example, lethally irradiating the recipient with selected levels of radiation, administering specific toxins to the recipient, administering specific monoclonal antibodies attached to toxins or radioactive isotopes, or combinations of these techniques. Certain embodiments of the invention contemplate only partial conditioning of the recipient as the donor cell dose is optimized.

In certain embodiments of the invention, bone marrow is harvested from the long bones of the donor. For allogeneic chimerism, donor and recipient are the same species; for xenogeneic chimerism, donor and recipient are different species. A cellular composition having T-cell depletion is described below. A separate cellular composition comprising a high concentration of hematopoietic progenitor stem cells is separated from the remaining donor bone marrow. Separation of a cellular composition comprising a high concentration of stem cells may be accomplished by techniques such as those used to purify FC, but based on different markers, most notably CD34 stem cell separation techniques. Such methods include those disclosed in U.S. Pat. No. 5,061,620 and the separate LC Laboratory Cell Separation System, CD34 kit manufactured by CellPro, Inc. (Bothell, Wash.). In certain embodiments, the purified donor facilitatory cell composition and purified donor stem cell composition are then mixed in any ratio. However, it is not necessary to mix these cellular compositions. The key is that if donor T-cells are not critical to engraftment one can find a way around them.

If the facilitatory cell is purified by negative selection using any or all of the markers disclosed herein not to be expressed on the facilitatory cell, then the resulting cellular composition will contain stem cells as well as FC and other immature progenitor cells. Antibodies directed to T-cell specific markers such as anti-αβ-TCR may be used to specifically eliminate GVHD-producing cells, while retaining hematopoietic facilitatory and stem cells without a need for substantial purification. In such a case, this one cellular composition may take the place of the two cellular compositions referred to hereinabove which comprise both purified FC and purified stem cells.

The purified donor FC and purified donor stem cells are then administered to the recipient. In certain embodiments, if these cellular compositions are separate compositions, they are administered simultaneously. However they may also be administered separately within a relatively close period of time. In certain embodiments, the mode of administration includes, but is not limited to, intravenous injection.

Once administered, it is believed that the cells home to various hematopoietic cell sites in the recipient's body, including bone cavity, spleen, fetal or adult liver, and thymus. The cells become seeded at the proper sites. The cells engraft and begin establishing a chimeric immune system. Since non-universal FC must be MHC-specific, as traditionally understood, with the stem cells whose engraftment they facilitate, it is possible that both the stem cells and FC bond together to seed the appropriate site for engraftment.

The level of alloengraftment or xenoengraftment is a titratable effect which depends upon the relative numbers of syngeneic cells and allogeneic or xenogeneic cells and upon the type and degree of conditioning of the recipient. Completely allogeneic or xenogeneic chimerism should occur if the FC of the syngeneic component have been depleted by TCD procedures or other techniques, provided that a threshold number of allogeneic or xenogeneic FC are administered; and the presence of T-cells to increase chimerism. A substantially equal level of syngeneic and allogeneic or xenogeneic engraftment is sought. The amount of the various cells that should be administered is calculated for a specific species of recipient. For example, in rats, the T-cell depleted bone marrow component administered is typically between about 1×10⁷ cells and 5×10⁷ cells per recipient. In mice, the T-cell depleted bone marrow component administered is typically between about 1×10⁶ cells and 5×10⁶ cells per recipient. In humans, the T-cell depleted bone marrow component administered is typically between about 1×10⁸ cells and 3×10⁸ cells per kilogram body weight of recipient. For cross-species engraftment, larger numbers of cells may be required.

In mice, the number of purified FC administered in certain embodiments is between about 1×10⁴ and 4×10⁵ FC per recipient. In rats, the number of purified FC administered in certain embodiments is between about 1×10⁶ and 30×10⁶ FC per recipient. In humans, the number of purified FC administered in certain embodiments is between about 1×10⁶ and 10×10⁶ FC per kilogram recipient.

In mice, the number of stem cells administered in certain embodiments is between about 100 and 300 stem cells per recipient. In rats, the number of stem cells administered in certain embodiments is between about 600 and 1200 stem cells per recipient. In humans, the number of stem cells administered in certain embodiments is between about 1×10⁵ and 1×10⁶ stem cells per recipient. The amount of the specific cells used will depend on many factors, including the condition of the recipient's health. In addition, co-administration of cells with various cytokines may further promote engraftment.

In addition to total body irradiation, a recipient may be conditioned by immunosuppression and cytoreduction by the same techniques as are employed in substantially destroying a recipient's immune system, including, for example, irradiation, toxins, antibodies bound to toxins or radioactive isotopes, or some combination of these techniques. However, the level or amount of agents used is substantially smaller when immunosuppressing and cytoreducing than when substantially destroying the immune system. For example, substantially destroying a recipient's remaining immune system often involves lethally irradiating the recipient with 950 rads (R) of total body irradiation (TBI). This level of radiation is fairly constant no matter the species of the recipient. Consistent xenogeneic (rat-mouse) chimerism has been achieved with 750 R TBI and consistent allogeneic (mouse) chimerism with 600R TBI. Chimerism was established by PBL typing and tolerance confirmed by mixed lymphocyte reactions (MLR) and cytotoxic lymphocyte (CTL) response.

As stated hereinbefore, the above disclosed methods may be used for establishing both allogeneic chimerism and xenogeneic chimerism. Xenogeneic chimerism may be established when the donor and recipient as recited above are different species. Xenogeneic chimerism between rats and mice, between hamsters and mice, and between chimpanzees and baboons has been established. Xenogeneic chimerism between humans and other primates is also possible. Xenogeneic chimerism between humans and other mammals is equally viable.

It will be appreciated that, though the methods disclosed above involve one recipient and one donor, the present invention encompasses methods such as those disclosed in which stem cells and purified FC from two donors are engrafted in a single recipient.

In certain embodiments, the present invention also provides methods of reestablishing a recipient's hematopoietic system by substantially destroying the recipient's immune system or immunosuppressing and cytoreducing the recipient's immune system, and then administering to the recipient syngeneic or autologous cell compositions comprising syngeneic or autologous purified FC and stem cells which are MHC-identical to the FC.

The ability to establish successful allogeneic or xenogeneic chimerism allows for vastly improved survival of transplants. The present invention provides for methods of transplanting a donor physiological component such as, for example, organs, tissue, or cells. Examples of successful transplants in and between rats and mice using these methods include, for example, islet cells, skin, hearts, livers, thyroid glands, parathyroid glands, adrenal cortex, adrenal medullas, and thymus glands. The recipient's chimeric immune system is completely tolerant of the donor organ, tissue, or cells, but competently rejects third party grafts. Also, bone marrow transplantation confers subsequent tolerance to organ, tissue, or cellular grafts which are genetically identical or closely matched to the bone marrow previously engrafted.

Transplanted donor organ, tissue, or cells competently perform their function in the recipient. For example, transplanted islet cells function competently, and thereby provide an effective treatment for diabetes. In addition, transplantation of bone marrow using certain methods of the present invention can eliminate the autoimmune diabetic trait before insulin-dependence develops. Successful solid organ transplants between humans and animals may be performed using certain methods of the present invention involving hematopoietic FC. For example, islet cells from other species may be transplanted into humans to treat diabetes in the human recipient after the disease is diagnosed or after the onset of insulin dependence. Major organs from animal donors such as, for example, pigs, cows or fish can solve the current problem of donor shortages. For example, 50% of patients who require a heart transplant die before a donor is available. It has been demonstrated that permanent acceptance of endocrine tissue engrafts (thyroid, parathyroid, adrenal cortex, adrenal medulla, islets) occurs in xenogeneic chimeras after bone marrow transplantation from a genetically identical donor. Hence, mixed xenogeneic chimerism or fully xenogeneic chimerism established by methods of the present invention can be employed to treat endocrine disorders as well as autoimmunity, such as, for example, diabetes.

Certain methods of the present invention include transplanting the specific donor physiological component by methods known to those skilled in the art and, in conjunction with establishing a chimeric immune system in the recipient, using the transplant donor as the donor of the purified donor facilitatory cell composition and donor stem cell composition.

Certain embodiments of the present invention comprise a mixed chimeric immune system. The method of establishing a mixed chimeric immune system may be performed before, during, or after the transplantation, but is preferably performed before the transplantation, especially since immunosuppression and cytoreduction or immunodestruction is necessary in the chimeric methods as disclosed herein. The methods disclosed allow for both allotransplantation and xenotransplantation. Because the methods disclosed herein provide for donor-specific immunotolerance, many procedures previously necessary to resist rejection of the donor organ, tissue, or cells are unnecessary. For example, live bone and cartilage may be transplanted by the methods disclosed herein.

Cell farming technology can provide for a readily available supply of FC, stem cells and genetically matched physiological donor components. For example, bone marrow cells enriched for the facilitatory cell can be propagated in vitro in cultures and/or stored for future transplantation. Cellular material from the same donor can be similarly stored for future use as grafts.

Beyond transplantation, the ability to establish a successful allogeneic or xenogeneic chimeric hematopoietic system or to reestablish a syngeneic or autologous hematopoietic system can provide cures for various other diseases or disorders which are not currently treated by bone marrow transplantation because of the morbidity and mortality associated with GHVD. Autoimmune diseases involve attack of an organ or tissue by one's own immune system. In this disease, the immune system recognizes the organ or tissue as a foreign. However, when a chimeric immune system is established, the body relearns what is foreign and what is self. Establishing a chimeric immune system as disclosed herein can simply halt the autoimmune attack causing the condition. Also, autoimmune attack may be halted by reestablishing the victim's immune system after immunosuppression and cytoreduction or after immunodestruction with syngeneic or autologous cell compositions as described hereinbefore. Autoimmune diseases which may be treated by this method include, for example, type I diabetes, systemic lupus erythematosus, multiple sclerosis, rheumatoid arthritis, psoriasis, colitis, and even Alzheimer's disease. The use of the FC plus stem cell can significantly expand the scope of diseases which can be treated using bone marrow transplantation.

It has recently been discovered that purified hematopoietic stem cells can differentiate into hepatocytes in vivo (Lagasse, E., et al., Nature Medicine, 6(11):1229-1234 (2000)). Accordingly, in certain other embodiments of the present invention FC can be added to stem cells to assist in the regeneration of organs and damaged tissues, including, but not limited to, heart tissue, skin, liver, lung, kidney, pancreatic tissue, organ, such as but not limited to, a thyroid gland, a parathyroid gland, a thymus, an adrenal cortex, an adrenal medulla.

Because a chimeric immune system includes hematopoietic cells from the donor immune system, deficiencies in the recipient immune system may be alleviated by a nondeficient donor immune system. Hemoglobinopathies such as sickle cell anemia, spherocytosis or thalassemia and metabolic disorders such as Hunters disease, Hurlers disease, and enzyme defects, all of which result from deficiencies in the hematopoietic system of the victim, may be cured by establishing a chimeric immune system in the victim using purified donor hematopoietic FC and donor stem cells from a normal donor. In certain embodiments, the chimeric immune system is at least 10% donor origin (allogeneic or xenogeneic).

In certain other embodiment of the invention, the ability to establish successful xenogeneic chimerism provides methods of treating or preventing pathogen-mediated disease states, including viral diseases in which species-specific resistance plays a role. For example, AIDS is caused by infection of the lymphohematopoietic system by a retrovirus (HIV). The virus infects primarily the CD4⁺ T-cells and antigen-presenting cells produced by the bone marrow stem cells. Some animals, such as, for example, baboons, possess native immunity or resistance to AIDS. By establishing a xenogeneic immune system in a human recipient with a baboon or other AIDS resistant and/or immune animal as donor, the hematopoietic system of the human recipient can acquire the AIDS resistance and/or immunity of the donor animal. Other pathogen-mediated disease states may be cured or prevented by such a method using animals immune or resistant to the particular pathogen which causes the disease. Some examples include hepatitis A, B, C, and non-A, B, C hepatitis. Since facilitatory cells play a major role in allowing engraftment of stem cells across a species disparity, this approach will rely upon the presence of the facilitatory cell in the bone marrow inoculum.

The removal of the facilitatory cell has been shown to substantially impair engraftment across species differences. However, while not the preferred approach, untreated xenogeneic bone marrow will engraft if sufficient cells are administered. Bone marrow-derived cells could be used in this case to treat or prevent AIDS with or without enrichment for the facilitatory cell. Previous studies demonstrated that GVHD could occur across a species barrier. Therefore, in various embodiments the approach would be to establish the xenogeneic chimeric immune system using cellular compositions comprising purified donor FC by methods disclosed herein or by using compositions depleted of T-cells.

Furthermore, some animals, such as, for example, baboons and other non-human primates, possess native immunity or resistance to hepatitis. By transplanting a liver from a baboon or other hepatitis resistant animal into a hepatitis patient using a method of the present invention, wherein a xenogeneic chimeric immune system is established in the victim using purified donor FC plus stem cells, the donor liver will not be at risk for hepatitis, and the recipient will be tolerant of the graft, thereby eliminating the requirement for nonspecific immunosuppressive agents. Unmodified bone marrow or purified stem cells may suffice as the liver may serve as a hematopoietic tissue and may contain FC that will promote the engraftment of stem cells from the same donor.

Establishing a mixed chimeric immune system has also been found to be protective against cancer (Sykes et al., Proc. Natl. Acad. Sci., U.S.A., 87: 5633-5637 (1990). Although the mechanism is not known, it may be due to multiplication of immune cell tumor specificity by the combination of donor and recipient immune system cells. In certain embodiments, a mixed chimerism system is utilized. However, fully allogeneic or fully xenogeneic chimerism may be utilized in certain instances. For example, the present invention includes methods of treating leukemia or other malignancies of the lymphohematopoietic system comprising substantially destroying the victim's immune system and establishing a fully allogeneic chimeric immune system by the methods described herein. Since the victim's own immune system is cancerous, in certain embodiments the syngeneic cells are fully replaced with allogeneic cells of a non-cancerous donor. In this case, autologous purified stem cells and FC may be used in order to totally eliminate all cancer cells in the donor preparation, especially if high dose chemotherapy or irradiation is used to ablate endogenous FC.

In certain embodiments, the present invention also provides methods of practicing gene therapy. It has recently been shown that sometimes even autologous cells which have been genetically modified may be rejected by a recipient. Utilizing methods of the present invention, a chimeric immune system can be established in a recipient using hematopoietic cells which have been genetically modified in the same way as genetic modification of other cells being transplanted therewith. This will render the recipient tolerant of the genetically modified cells, whether the recipient is autologous, syngeneic, allogeneic or xenogeneic.

It will be appreciated that the present invention discloses cellular compositions comprising purified FC cellular compositions depleted of T-cells with the retention of FC and stem cells, methods of purifying FC, methods of establishing fully, completely or mixed allogeneic or xenogeneic chimeric immune systems, methods of reestablishing a syngeneic immune system, and methods of utilizing compositions of FC to treat or prevent specific diseases, conditions or disorders. It will also be appreciated that the present invention discloses methods of treating or preventing certain pathogen-mediated diseases by administering xenogeneic cells which have not been purified for the facilitatory cell.

B. FC Function to Regulate HSC Survival and Function via TNF-α and Bcl-3

In the present invention, the possibility that FC function is mediated by TNF-α secretion was also investigated. Since TNF-α is a potent effector on HSC, and has been shown to increase survival of quiescent HSC (Snoeck, 1996 2836), it was hypothesized that FC act on HSC via TNF-α Using TNF-α deficient mice as FC donors, it is demonstrated herein that graft FC enhance HSC engraftment in syngeneic and allogeneic recipients via a TNF-mediated mechanism. More specifically, the present teachings demonstrate that FC require TNF-α to facilitate HSC engraftment in allogeneic and in syngeneic recipients in vivo, as well as in their role of enhancing HSC clonogenicity measure in vitro.

The present invention further demonstrates that preincubation of FC with anti-TNF antibody inhibits the effect of wild type FC on HSC clonogenicity and survival, confirming a role for TNF-α in these FC functions. Furthermore, the present teaching show that neutralization of TNF-α on FC also blocked upregulation of Bcl-3 transcripts in HSC. Together, these data demonstrate the role of TNF-α, at least in part via Bcl-3, in FC function in vivo and in vitro.

Accordingly, certain embodiments of the present invention comprise a cellular composition comprising human hematopoietic stem cells (HSC), which are depleted of graft-versus-host-disease-producing cells having a phenotype of αβ-TCR⁺ and which have retained mammalian hematopoietic facilitatory cells having a phenotype of CD8⁺/TCR⁻, CD8⁺/TCR⁺, which facilitatory cells have an increased expression of TNF-α and/or increase ability to upregulate Bcl-3 transcription in said HSC compared to wild-type FC, and which FC are capable of facilitating engraftment of bone marrow cells.

The present invention further provides methods for conditioning a recipient for bone marrow transplantation using the compositions of this invention. More specifically, certain embodiment of this invention provide methods for conditioning a recipient for bone marrow transplantation comprising introducing CD8⁺/TCR⁻ facilitating cells and purified hematopoietic stem cells into a recipient lacking T-cells, and stimulating the expression of TNF-α in FC and/or increasing the uptake of Bcl-3 by HSC. In certain embodiments, expression of TNF-α and/or uptake in Bcl-3 is increased by introducing to the FC an agonist that stimulates the expression of TNF-α and/or uptake in Bcl-3.

In certain embodiments, the stimulation occurs prior to the introduction of the HSC to the recipient. In certain other embodiments, the stimulation occurs subsequent to introduction of the HSC to the recipient.

As used herein, “recipient” includes any mammalian subject, such as human, to which a composition of this invention is administered, and includes allogeneic and syngeneic recipients.

Methods of increasing expression of TNF-α or uptake in Bcl-3 include introducing to the FC an agonist that increases expression of TNF-α or uptake of Bcl-3 by HSC.

FC from TNF-α^{-/-} mice were significantly impaired in function in vivo as evidenced by loss of facilitative capability and in vitro in enhancing stem cell clonogenicity. Pretreatment of FC from TNF-α^{-/-} mice with TNF-α transiently restored the ability of FC to increase CFC. However, the effect disappeared after 18 hours of incubation, possibly due to the membrane uptake of the TNF-α, which is then consumed and therefore no longer detectable. These data confirm a role for TNF-α in FC function, as well as a regulatory role for FC on HSC via TNF-α.

TNF-α has been shown to be a bifunctional regulatory of hematopoietic stem and progenitor cells, depending upon the growth factors used as supplements, as well as the maturation stage of the cell (Zhang, 1995). Most notably, TNF-α inhibits the growth of primitive HSC, preventing entry into s-phase from G₀/G₁ during stimulation by growth factors (Zhang, 1995).

It is demonstrated in the present invention that HSC stimulate FC to produce TNF-α at the mRNA and protein levels, although at very low quantities (under the limit of detection of cytokine array<3 pg/mL), and possibly mostly in membrane form. Several studies have demonstrated a dose dependant effect of TNF-α on hematopoietic cells (Maguer-Satta, 2000; Broxmeyer, 1986; Rusten, 1994). It is also possible that the TNF-α produced by FC when in contact with HSC is consumed immediately and is thus more difficult to detect. The fact that the supernatant from an 18 hour culture of FC plus HSC increases HSC clonogenicity, and that this effect can be inhibited in most cases by blocking TNF with anti-TNF-α antibody on FC, implies the presence of soluble TNF-α. However, the inhibition was partial, suggesting the presence of other efficient factors such as IL-6 (also produced by FC), which is a good candidate for activating or priming HSC for CFC. Conversely, supernatants from FC+HSC cultures failed to increase HSC survival. This could be due to the need for direct cell contact between FC and HSC if the predominant form of TNF-α were membrane-bound, or perhaps a requirement for a higher TNF-α concentration in the supernatant. The fact that FC lose their ability to increase HSC survival when separated by a membrane in culture (data not shown) seems to favor the former, that FC need to be in contact with HSC for the anti-apoptotic effect. Furthermore, it has been suggested that the effect of cell-bound TNF-α is advantageous for the host (Kriegler, 1988; Kinkhabwala, 1990) which tends to corroborate the observations disclosed herein.

The role of TNF-α in the ability of FC to increase HSC clonogenicity was further confirmed by the fact that pretreatment of FC with anti-TNF-α neutralizing antibodies completely inhibited the effect of FC on HSC clonogenicity. This observation contradicts some previous reports (Broxmeyer, 1986; Maguer-Satta, 2000; Skobin, 2000). However, the very low dose and the short exposure of TNF-α presented by FC and/or in the milieu may have a different outcome. The model disclosed herein differs from prior models in that an accessory cell is utilized rather than cocktails of cytokines to examine the role of TNF-α in maintaining HSC quiescence. The teachings of the present invention are in correlation with previous observations on human CD24⁺/CD38⁻ stem cells, where the maximal effect of TNF-α on proliferation and CFC was at doses between 0.5 and 2.5 ng/mL (Snoeck, 1996). It is also possible that FC produce a combination of cytokines which synergistically act on HSC to provide that effect (Snoeck, 1996).

The present teachings demonstrate that the production of TNF-α by FC results in upregulation of Bcl-3 in HSC, since blocking of TNF-α on FC abrogated this effect. Notably, it has been shown that the anti-apoptotic effect of FC was associated with significant upregulation of Bcl-3 transcription in the HSC. Bcl-3 is a member of the IκB subfamily of transcriptional regulators which typically function by retaining the DNA-binding members of the NFκB family in inactive complexes in the cytoplasm (Kim, 2002). Bcl-3 is an atypical member of this family in that it can increase rather than inhibit the transcriptional activity of NFκB family members (Kerr, 1992). Preincubation of FC with anti-TNF-α prior to co-culture with HSC totally abrogated Bcl-3 transcription in HSC. Alternatively, incubation of HSC with TNF-α stimulated Bcl-3 transcription in HSC in the same manner as FC, confirming the role of TNF-α in this pathway. The bifunctional regulatory effect of TNF-α is well known. On one hand, it is an apoptotic agent for HSC, as well as for many other cell types. On the other hand, it has been shown to activate NF-κB, allowing cells to escape apoptosis (Beg, 1996; Pyatt, 1999; Kerzic, 2003). NF-κB is present in all human bone marrow cells and is required for survival as well as for clonogenicity of HSC (Pyatt, 1999). The present teachings suggest a role for TNF-α produced by FC in HSC survival by regulating NF-κB activity through at least Bcl-3. This is in agreement with previous observations, showing that TNF-α protect quiescent stem cells from apoptosis (Bryder, 2001; Ratajczak, 2003). In addition, this can explain the previous observations of FC increasing HSC survival and also the frequency of primitive stem cells within the HSC. The exact role of Bcl-3 in HSC survival regulation has still to be established. The fact that HSC stimulate FC to produce TNF-α also confirms previous suggestions of a cross talk (activation) between HSC and FC. In the apoptosis assay, co-culture of HSC with FC rescued wild type FC but not TNF-α^{-/-} FC from apoptosis (data not shown), further confirming the cross-talk between these two cell populations via TNF-α. It is believed that this is the first demonstration of a specific cell in the hematopoietic microenvironment to mediate this effect.

Finally, to further evaluate the mechanism of action of FC and TNF-α on HSC engraftment, HSC from mice deficient for both TNF-α receptors p55 and p75 were used. It has previously been shown that HSC from TNFR p55^{-/-} mice are impaired in their self-renewal ability, implicating an important role for TNF-α signaling via the p55 receptor pathway for inhibition of growth of both primitive stem and progenitor cells (Rebel V I, et al., J. Experimental Medicine 1999;190:1493-1504; Zhang, 1995). In the allogeneic model described herein, TNFR^{-/-} HSC show low engraftment efficiency, confirming these previous observations. Co-injection of FC with TNFR^{-/-} HSC significantly increased TNFR^{-/-} HSC short-term engraftment for up to 180 days with a donor engraftment of 60% at 3 months. However, survival rapidly declined thereafter. The lack of effect of WT FC on TNFR^{-/-} HSC clonogenicity suggests that TNF-α is more likely involved in regulating the long-term repopulating function of HSC than in the early differentiation for radioprotection. The role of FC would be to provide the necessary regulatory environment for HSC to efficiently function after transplantation.

Although the regulation of survival and proliferation of HSC is complex and involves several pathways, it has been demonstrated herein that FC, in part by producing TNF-α, directly affect HSC function and survival, and that this is associated with upregulation of Bcl-3 transcription. This finding further identifies the regulatory role of accessory cells such as FC in the bone marrow microenvironment, the interrelation between HSC and FC in the bone marrow, and the requirement to transplant them together with HSC in order to enhance the graft success.

The present invention thus includes, but is not limited to, the following compositions and methods:

1. A cellular composition comprising mammalian hematopoietic stem cells, such as human hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells, wherein said facilitatory cells have an increased expression of TNF-α and/or an increased ability to upregulate Bcl-3 in said hematopoietic stem cells relative to wild-type facilitatory cells.

In various embodiment, the cellular composition further comprises a pharmaceutical composition that increases expression of TNF-α from said facilitatory cells and/or increases the ability of said facilitatory cells to upregulate said Bcl-3.

2. A method of partially or completely reconstituting a mammal's lymphohematopoietic system comprising:

(a) administering to the mammal a cellular composition comprising mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and

(b) stimulating the expression of TNF-α from said facilitatory cells and/or increasing the ability of said facilitatory cells to upregulate Bcl-3 in said hematopoietic stem cells.

In certain embodiments, said stimulation comprises introducing to said facilitatory cells a pharmaceutical composition that increases TNF-α expression.

In certain embodiments, said increase in upregulation of Bcl-3 is effected by introducing to said facilitatory cells a pharmaceutical composition that increases said upregulation.

In certain embodiments, the mammal is conditioned by total body irradiation.

In certain embodiments, said mammal is conditioned by an immunosuppressive agent.

In certain embodiments, said mammal is conditioned by a cytoreduction agent.

In certain embodiments, said mammal is a human.

In certain embodiments, said mammal suffers from autoimmunity. Examples of autoimmunity include, but are not limited to, diabetes, multiple sclerosis, lystemic lupus erythematosus, systemic sickle cell.

In certain embodiments, said mammal suffers from immunodeficiency.

In certain embodiments, said mammal is infected with a human immunodeficiency virus.

In certain embodiments, said mammal is infected with a hepatitis virus.

In certain embodiments, said mammal suffers from a hematopoietic malignancy.

In certain embodiments, said mammal suffers from anemia.

In certain embodiments, said mammal suffers from hemoglobinopathies.

In certain embodiments, said mammal suffers from an enzyme deficiency state.

In certain embodiments, said mammal is human and the cellular composition is obtained from a human.

In certain embodiments, said mammal is human and the pharmaceutical composition is obtained from a non-human animal.

In certain embodiments, said mammal requires a solid organ or cellular transplant.

3. A method of inducing tissue or organ regeneration in a mammal comprising: (a) administering to the mammal a cellular composition comprising mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and

(b) stimulating the expression of TNF-α from said facilitatory cells and/or increasing the ability of said facilitatory cells to upregulate Bcl-3 in said hematopoietic stem cells.

Examples of said donor organ include, but are not limited to, heart, skin, liver, lung, heart and lung, kidney, pancreatic islet cells or whole pancreas, endocrine organ, a thyroid gland, a parathyroid gland, a thymus, adrenal cortex, adrenal medulla, and neurons, myocytes.

4. A method of increasing HSC engraftment efficiency, comprising co-incubating prior to engraftment a pharmaceutical composition that stimulates TNF-α expression and a cellular composition comprising human hematopoietic stem cells, wherein said stem cells are depleted of graft-versus-host-disease-producing cells having a phenotype of αβTCR+ with the retention of mammalian hematopoietic facilitatory cells having a phenotype of CD8+/TCR+, CD8+/TCR−, which hematopoietic facilitatory cells are capable of facilitating engraftment of bone marrow cells.

Whereas particular embodiments of the invention has been described hereinbefore, for purposes of illustration, it would be evident to those skilled in the art that numerous variations of the details may be made without departing from the invention as defined in the appended claims.

The invention is discussed in more detail in the subsections below, solely for the purpose of description and not by way of limitation. For clarity of discussion, the specific procedures and methods described herein are exemplified using a murine model; they are merely illustrative for the practice of the invention. Analogous procedures and techniques are equally applicable to all mammalian species, including human subjects.

### EXAMPLES

### Materials and Methods

Animals:

Five-to seven-week-old male ACI (RT1Aa), Wistar Furth (WF; RT1Au), and Fisher (F344; RT1A1) rats were purchased from Harlan Sprague Dawley (Indianapolis, Ind.). Animals were housed in a barrier animal facility at the Institute for Cellular Therapeutics, University of Louisville, Louisville, Ky., and cared for according to specific University of Louisville and National Institutes of Health animal care guidelines.

TCD of Bone Marrow in vitro:

TCD was performed as described previously. Briefly, bone marrow was harvested from femurs and tibias of ACI rats by flushing with Media 199 (GIBCO, Grand Island, N.Y.) containing 2 μg/mL gentamicin (MEM), using a 22-gauge needle, and then filtered through sterile nylon mesh. Bone marrow cells were washed, counted and resuspended to 100×10⁶ cells/mL in 1× Hanks' balanced salt solution containing 10% fetal bovine serum. Cells were incubated with anti-αβ-TCR monoclonal antibody (mAb) (R73; mouse IgG1; Pharmingen, San Diego, Calif.) and/or anti-γδ-TCR mAb (V65; mouse IgG1; Pharmingen) for 30 min at 4° C. The cells were washed twice to remove unbound primary mAb and incubated for 60 minutes at 4° C. with Dynabeads M-450 (goat anti-mouse IgG) immunomagnetic beads at a bead/T-cell ratio of approximately 20:1. T-cells were then isolated from bone marrow by magnetic separation and the unbound bone marrow cells were removed with the supernatant. TCD-bone marrow cells were resuspended in MEM at a final concentration of 100×10⁶ cells/mL.

Verification of TCD by Flow Cytometry:

To confirm adequacy of TCD, pre-depletion cells, post-incubation cells, and post-depletion cells were incubated with anti-αβ-TCR-fluorescein isothiocyanate (FITC), anti-γδ-TCR-phycoerythrin (PE) or rat adsorbed goat anti-mouse Ig-FITC (Pharmingen), the secondary antibody for αβ-TCR or γδ-TCR for 30 min. The latter stain detects coating and saturation of the target cells with mAbs. These cells were also incubated with anti-CD8-FITC, anti-CD3-PE, and biotinylated anti-αβ-TCR and streptavidin-conjugated antigen presenting cells (APQ (Phanningen) to enumerate CD3⁺ and CD8⁺ cell populations. Facilitating cells were enumerated using two- and three-color flow cytometry to detect CD3⁺/CD8⁺/TCR⁻ cells. Then cells were washed twice in “fluorescence-activated Cell Sorter” (FACS) medium (prepared in laboratory) and fixed in 1% paraformaldehyde (Tousimis Research Corporation, Rockville, Md.). Flow cytometric analysis was performed on a FACSCalibur (Becton Dickinson, Mountain View, Calif.).

Preparation of Mixed Allogeneic Chimeras (ACI→WF):

Mixed allogeneic chimeras were prepared by methods previously described by the inventor. Briefly, Wistur-Furth (WF) rats were conditioned with 950 cGy of TBI. Using sterile technique, recipients were reconstituted within 4-6 hours following TBI with 100×106 TCD bone marrow cells from ACI rats diluted in 1 mL MEM via penile vein injection. Control WF rats received equal numbers of untreated bone marrow cells.

Determination of Chimerism:

Thirty days post-BMT, recipients were characterized for allogeneic engraftment using two-color-flow cytometry. Chimerism was determined measuring the percentage of peripheral blood lymphocyte (PBL) of ACI or WF MHC class I antigen. Briefly, whole blood of recipients was collected in heparinized tubes, and aliquots of 100 μL were stained with purified anti-RTIAu (NR3/31; rat IgG2a; Serotec, Toronto, Ontario, Canada) and biotinylated anti-RTIAa,b (C3; LOU/cN JgG2b; Pharmingen) mAbs for 30 minutes. The cells were washed twice, then counterstained with anti-rat IgG2a -FITC (RG7/1.30; mouse IgG2b, Pharmingen) or streptavidin-conjugated antigen presenting cells (APC) (Pharmingen). Red blood cells were lysed with ammonium chloride lysing buffer for 5 minutes at room temperature. The cells were then washed in FACS medium and fixed in 1% paraformaldehyde.

Assessment of GVHD:

All chimeras were evaluated for manifestations of GVHD on a daily basis for the first month following reconstitution and weekly thereafter. The primary diagnosis of GVHD was based on previously described clinical criteria, which consist of diffuse erythema (particularly of the ear), hyperkeratosis of the foot pads, dermatitis, weight loss, generalized unkempt appearance, or diarrhea. An animal was considered to exhibit acute GVHD if at least four of the above signs were observed. The diagnosis of GVHD was confirmed by the histologic analysis of skin, tongue, liver, and small intestine following 30, 60, 90, 150, or 220 days. Tissues were fixed in 10% buffered formalin for routine hematoxylin and eosin (H&E) staining. Grading of GVHD was performed in a blinded fashion according to previously described histologic criteria.

Intra-Abdominal Heterotopic Cardiac Transplantation:

Four months after “bone marrow transplantation” (BMT), cardiac allografts from ACI, WF, and F344 rat donors were transplanted into mixed allogeneic chimeras as previously described. Allograft survival was assessed daily, based on the presence and quality of the graft heartbeat graded from 0 (no palpable beat) to 4 (visual pulsation).

Rejection of cardiac allografts was defined as cessation of visible or palpable cardiac contractions and was confirmed by the histologic presence of a mononuclear cell infiltrate and myocyte necrosis on H&E stained sections.

Statistical Analysis:

Experimental data were evaluated for significant differences using (a) the Independent Sample test; P<0.05 was considered significant difference, or (b) the one tail-paired Student t-test. Data were considered significantly different when the probability value was less than 0.05 (P<0.05). Graft survival was calculated according to the Kaplan-Meier method.

Animals and Transplantation Procedure:

Four to six week old C57BL/6JEi (B6, H-2^{b}) and B10.BR (BR, H-2^{k}) mice were purchased from the Jackson Laboratory (Bar Harbor, Maine). TNF-α^{-/-} and TNF-α receptor^{-/-} (TNF-αR) deficient mice (H-2^{b}) were bred at the Institute for Cellular Therapeutics. Mice were used at 4-12 weeks of age. Animals were housed in the barrier animal facility at the Institute for Cellular Therapeutics, University of Louisville, Louisville, Ky., and cared for according to specific guidelines per National Institutes of Health.

Cell Preparation:

HSC, FC and T-cells were isolated from bone marrow (BM) by multiparameter, live sterile cell sorting (FACSVantage SE, Becton Dickinson, Mountainview, Calif.), as previously described (Fugier-Vivier, et al., J. Exp. Med. 2004, 4892, In press, Huang, 2004, 4637). Briefly, BM cells were isolated in HANK'S balanced salt solution (HBSS, Gibco, Grand Island, N.Y.) supplemented with 2% heat-inactivated fetal bovine serum (FBS, Gibco), 10 mM HEPES buffer (Gibco) and 50 μg/mL Gentamicin (Gibco). For HSC isolation, the BM cell suspension was lysed of red cells using ammonium chloride solution (ACK), washed, and incubated 30 minutes on ice with monoclonal antibodies (mAb) against: Sca-1-phycoerythrin (PE)-labeled, CD117 (c-kit)-Allophycocyanin (APC)-labeled and fluorescein isothiocyanate (FITC)-labeled anti-lineage mAb: B220 (CD45 RB), CD11b (Mac-1), Gr-1, TCRβ, TCR-γδ, or with isotype-matched control antibodies. Cells were washed and sorted for c-kit⁺/Sca-1⁺/Lin⁻ (KSL) expression. For FC and T-cell sorting, BMC were incubated with anti-CD8α PE, anti-TCRβ and anti-TCRγδ FITC or with isotype-matched control antibodies. FC were sorted for CD8⁺/TCRαβ⁻/TCRγδ⁻ expression and T-cells for CD8⁺/TCRαβ⁺/TCRγδ⁺ expression. All antibodies were purchased from Becton Dickinson/PharMingen (San Diego, Calif.).

Transplantation:

Recipient mice received total body irradiation (TBI) at 950 cGy (Cesium source, ¹³⁷Cs, Gamma-cell 40, Nordion, Ontario, Canada) and were transplanted by tail vein injection at least 6 hours after irradiation. Syngeneic reconstitution: 500 B6 HSC in the presence or absence of 30×10³ FC were transplanted into conditioned B6. Allogeneic reconstitution: conditioned B10.BR mice were transplanted with 10×10³ B6 HSC in the presence or absence of 30×10³ FC or with 10×10³ HSC from TNF receptor^{-/-} mice in the presence of 30×10³ B6 FC. Sorted cells were mixed before transplantation.

Cell Culture:

15×10³ HSC alone or plus 30×10³ FC from B6 or from TNF-α^{-/-} mice were incubated for 18 or 40 hours in 200 μL long-term culture media (LTCM, Iscove modified Eagle's Medium (IMDM, Gibco) supplemented with 20% horse serum (Gibco)), 10⁻⁶M hydrocortisone (Sigma, St Louis, Mo.), 10⁻⁵ M β-mercaptoethanol (Sigma), 100 U/mL penicillin, 100 μg/mL streptomycin (Gibco), 2 mM L-glutamine (Gibco) and 25 mM NaHCO₃ (Sigma) in a 96 well round bottom plate and at 37° C.-5% CO₂ in a humidified atmosphere. In some experiments, HSC (15×10³) were incubated for 18 hours with 10 ng/mL of TNF-α or with a 1/10 dilution of conditioned media (SN, from 18 hour culture of FC+HSC). After incubation, 100 μL supernatant (SN) was removed and frozen for further analysis, cells were resuspended and used in the CFC assay. Some of the SN was used in the CFC assay as well, or for the analysis of the presence of cytokines.

In experiments with anti-TNF-α, FC were preincubated for 1 hour with anti-TNF-α mAb or isotype control, and then co-cultured as described with HSC. In experiments with TNF-α^{-/-} FC, FC were preincubated with TNF-α (10 ng/mL) for 1 hour, washed once in culture media to remove the excess antibody and co-cultured with HSC as described above.

Colony-Forming Cell Assay (CFC):

CFC was performed on freshly sorted cells or on cells after 18 or 40 hour incubations. HSC were suspended at 100 cells/mL in methylcellulose containing mouse growth factors (MethoCult GF M3434, StemCell Technologies, Vancouver, BC, Canada) and plated at 500 μL (50 HSC)/well in 24 well plates in duplicate. Cultures were incubated at 37° C.-5% CO₂ for 14 days, at which time colonies containing more than 50 cells were scored using an inverted microscope.

Supernatant Preparation and Evaluation of Cytokines and Chemokines:

HSC (30×10³) were incubated alone or in the presence of 100×10³ FC or T-cells. As a control, FC or T-cells were incubated alone or with TLR-9 ligand CPG ODN 1668 (TCCATGACGTTCCGATGCT) from Gibco BRL Custom Primers at 1 μM. Supernatants were collected 18 hours later and stored at −20° C. for further analyses. Evaluation of cytokines and chemokines present in the supernatant has been performed by Linco diagnostic using LINCOplex™ Multiplex Immunoassay for mouse cytokines (Linco Diagnostic, St Charles, Mo.), and three supernatants of each culture type have been analyzed in duplicates with data reported in pg/mL.

Cell Surface and Intracellular Staining for TNF-α

The cells remaining in the well from the supernatant production described above were analyzed for the presence of TNF-α. For surface TNF-α, anti-TNF-α-PE mAb was added to the cell suspension. After a 30 minute incubation on ice, cells were washed in FACS media: HBSS containing 0.1% BSA (bovine serum albumin, Sigma), 0.1% sodium azide (Sigma) and 0.04% sodium bicarbonate (Sigma), and then analyzed by four-color flow cytometry on a FACSCalibur (Becton Dickinson) using CellQuest software (Becton Dickinson).

For intracellular staining of TNF-α in FC or T-cells, Brefeldin A at 10 μg/mL (GolgiStop®, BD/Pharmingen) was added directly in the well 4 hours before the staining and incubated at 37° C., followed by an incubation with Fc γblock (anti-CD16/CD32 antibodies) for 10 minutes and another wash in FACS media. Cells were fixed with 4% formaldehyde in FACS media for 15 minutes on ice, then permeabilized with Saponin buffer (0.1% Saponin in FACS media) for 10 minutes and stained with anti-TNF-α-PE (diluted in Saponin buffer) for 25 minutes on ice. Cells were analyzed as described for surface staining.

Apoptosis Assay

Sorted HSC were incubated as described before at 15×10³ cells/well in a 96 well round bottom plate, alone or in the presence of 45×10³ B6 FC or FC from TNF-α^{-/-} mice for 18 or 40 hours in complete RPMI (Gibco) with 2% FBS (HyClone, Logan, Utah), in duplicate. In some experiments, FC were first incubated with anti-TNF-α mAb for 1 hour before being co-cultured with HSC. After incubation, cells were blocked with anti-CD16/CD32 Ab for 15 minutes at room temperature in order to avoid nonspecific staining, then stained with APC labeled c-kit mAb to identify stem cells, and simultaneously assayed for apoptosis by using 2 μL Annexin V-FITC (PharMingen). Both c-kit and Annexin V staining took place in the well for 20 minutes at room temperature. Cell death was measured using 2 μL of a 125 μg/mL working dilution of 7AAD (7-aminoactinomycin D; Molecular Probe, Eugene, Oreg.) added 1 minute before flow cytometry analysis. Lymphoid-gated, c-kit⁺ cells were separated into three categories based on Annexin V and 7AAD staining patterns.

Real Time Reverse Transcription Polymerase Chain Reaction (RT-PCR) on Cultured Cells:

Culture conditions and cell re-sort: HSC were incubated at 30×10³ per well in a 96 well round bottom plate for 16 hours in the presence or absence of 90×10³ FC or TNF-α (10 ng/mL) in the same conditions described for the apoptotic assay. In some experiments, FC were preincubated with anti-TNF-α mAb for 1 hour before being co-cultured with HSC. After incubation, FCγ were blocked and cells were labeled with c-kit APC as already described, then placed in tubes with 200 μL HBSS (Gibco) and sorted again on a FACSVantage for c-kit⁺ cells (HSC) and c-kit⁻ cells (FC).

Real time RT-PCR assay: total mRNA was isolated from cells with the RNeasy Mini Kit (Quiagen Inc, Valencia, Calif.) and reverse-transcribed with TaqMan Reverse Transcription Reagents (Applied Biosystems, Foster City Calif.). Detection of Bcl-x_{L}, Bax, FLIP, Bcl-2, Bcl-3, p53, CXCR4, TNF-α and β2 microglobulin mRNA levels was performed by real-time RT-PCR using an ABI PRISM® 7000 Sequence Detection System (ABI, Foster City, Calif.). Each 25 μL reaction mixture contained 12.5 μL SYBR Green PCR Master Mix, 10 ng of cDNA template and primer mRNA. Primers mRNA were as follows: 5′-AGG CAG GCG ATG AGT TTG AAC-3-forward and 5′-CCT GCT CAA AGC TCT GAT ACG C-3′ reverse for Bcl-x_{L}; 5′-TGG ATA GCA ATA TGG AGC TGC A-3′ forward and 5′ TGC CAT CAG CAA ACA TGT CAG-3′ reverse for Bax; 5′-TAT GCA AGT ATG GCC CAA CAT C-3′ forward and 5′-GAG TGA ACT TGA TCT CTG CCC A-3′ reverse for FLIP; 5′-CAG AGA TGT CCA GTC AGC TGC A-3′ forward and 5′-AAA GAA GGC CAC AAT CCT CCC-3′ reverse for Bcl-2; 5′-ATA TCA GCC TCG AGC TCC CTC T-3′ forward and 5′-ATC GTC CAT GCA GTG AGG TGA-3′ reverse for p53; 5′-TGC AGT GGA TAT CAA GAG CGG-3′ forward and 5′-ACA TCT GAG CGT TCA CGT TGG-3′ reverse for Bcl-3; 5′-GGA TGC AGA AGG AGA TCA CTG-3′ forward and 5′-CGA TCC ACA CGG AGT ACT TG-3′. Add TNF and CXCR4 reverse primers for β2 microglobulin. Primers were designed with the Primer Express software. The threshold cycle (Ct), i.e., the cycle number at which the amount of amplified gene of interest reached a fixed threshold, was determined subsequently. Relative quantitation of Bcl-x_{L}, Bax, FLIP, Bcl-2, Bcl-3, p53, and TNF-α mRNA expression was calculated with the comparative Ct method described elsewhere (Livak, 2001). The relative quantitation value of target, normalized to an endogenous control β2 microglobulin gene and relative to a calibrator, is expressed as 2^{-ΔΔCt} (fold difference), where ΔCt=Ct of target genes (Bcl-x_{L}, Bax, FLIP, Bcl-2, Bcl-3, p53, and TNF-α)—Ct of endogenous control gene (β2 microglobulin), and ΔΔCt=ΔCt of samples for target gene-ΔCt of calibrator for the target gene.

### Example 1

### Depletion of αβ- and γδ-TCR⁺ T-Cells from Rat Marrow does not Remove FC

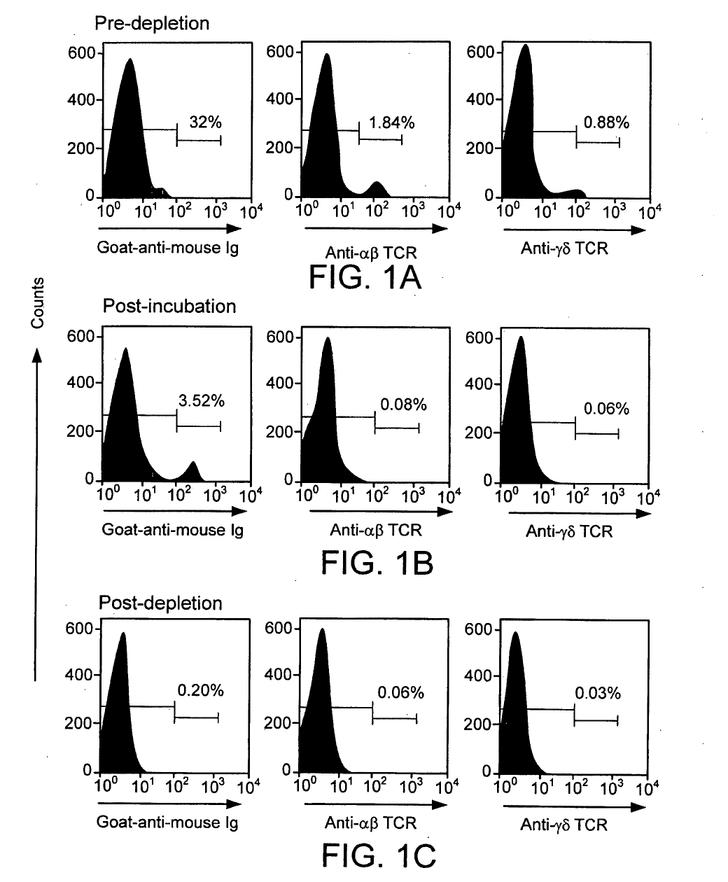

αβ- and γδ-TCR⁺ T-cells comprise 2% to 4% of the rat marrow. TCD of ACI marrow reduced the proportion of αβ-TCR⁺ T-cells from 1.84%±0.99% to 0.06%±0.03%, and γδ-TCR⁺ T-cells from 0.88%±0.32% to 0.03%±0.02% (Table 1). FIG. 1 illustrates T-cell depletion of rat bone marrow. Adequacy of αβ- and γδ-TCR⁺ T-cell depletion was confirmed using anti-αβ-TCR FITC and anti-γδ-TCR PE or rat adsorbed goat anti-mouse Ig FITC mAbs pre-depletion (A), post-incubation (B) and post-depletion (C). Staining with these mabs demonstrated that αβ- and γδ-TCR⁺ T-cells had been effectively depleted.

TABLE 1
Efficacy of T-cell depletion was confirmed by flow cytometry

| | |
|---|---|
| Cells depleted | |
| from | % T-cell of bone marrow (mean ± SD)^{a} |

| Donor marrow | Pre-depletion | Post-depeltion |
|---|---|---|
| αβ-TCR | 1.84 ± 0.99 | 0.06 ± 0.03 |
| γδ-TCR | 0.88 ± 0.32 | 0.03 ± 0.02 |
| αβ- and γδ-TCR | 3.40 ± 1.29 | 0.07 ± 0.01 |

^{a}Results are expressed as the mean ± SD of at least 4 experiments

Efficacy of TCD was confirmed by flow cytometry. The adequacy of depletion was further confirmed using goat anti-mouse Ig, an isotype and species-specific secondary antibody for the anti-αβ-TCR or γδ-TCR mAbs which would enumerate cells that were coated with antibody but not removed (FIG. 1, left column).

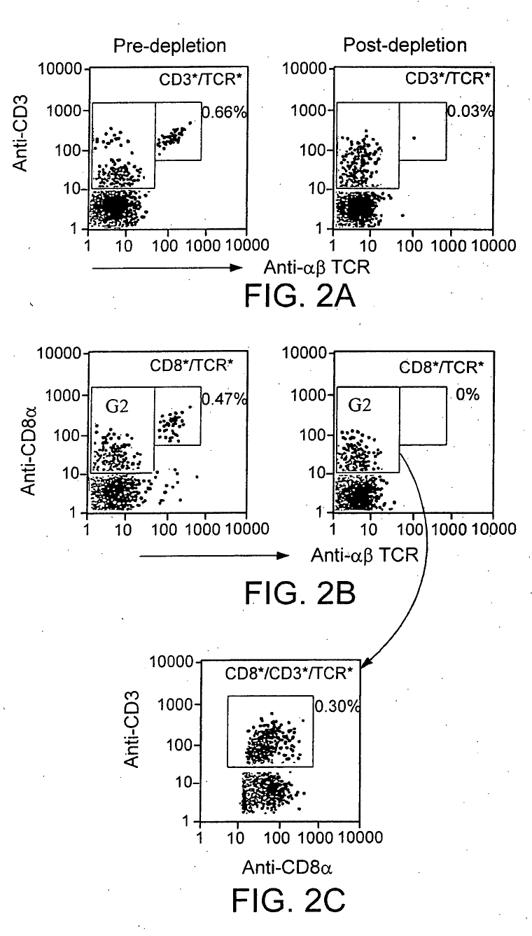

FIGS. 2A, 2B, and 2C illustrate the detection of facilitating cells. Bone marrow cells (pre- and post-depletion) were analyzed for the presence of facilitating cells using three-color-flow cytometry. Staining using anti-CD8a FITC, anti-CD3 PE and anti-αβ-TCR biotin (sandwiched with streptavidin APC mAbs showed that CD3+/CD8+/TCR⁻ cell population remains in marrow after depletion of αβ- and γδ-TCR⁺ T-cells. A and B, bone marrow cells were analyzed for their CD8, CD3 and TCR expression from lymphoid gate (G1) and CD8⁺/TCR⁻ were gated (G2). C, CD8⁺/CD3⁺/TCR⁻ cells remain in marrow after TCD (from G2). A minimum of 100,000 events was counted. As indicated in FIG. 2, the FC population (CD8⁺/CD3⁺/TCR⁻) is still present in marrow after depletion of αβ-and γδ-TCR⁺ T-cells at a level ranging from 0.23% to 0.45% of total cells. The CD8^{bight} population of conventional T-cells was removed while the CD8^{intermediate}/TCR⁻ FC population remained.

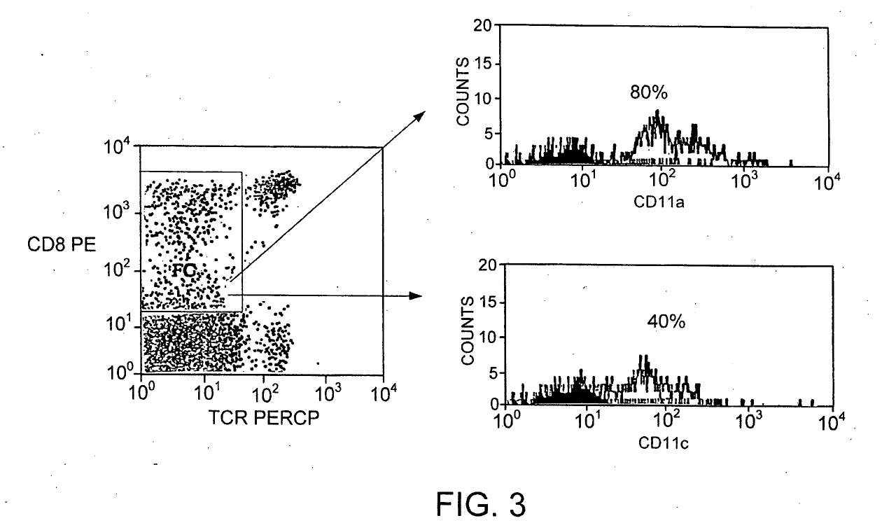

The CD8⁺/TCR⁻ FC population was also analyzed for expression of CD11a and CD11c. A minimum of 100×10³ events were analyzed. CD11a is expressed on macrophages, on monocytes, and is a developmental marker on lymphocytes. CD11b is expressed primarily on macrophages and monocytes, while CD11c is predominantly expressed on dendritic cells. Approximately 40% of the CD8⁺/TCR7 FC are CD11c⁺ (FIG. 3). Thirty-five percent of FC cells were also positive for the dendritic cell marker OX-62. CD11a was expressed on 80% of FC. The percentage of CD11a and CD11c positive cells were based on the FC gate.

### Example 2

### Depletion of αβ- and γδ-TCR⁺ T-Cells from Donor Marrow does not Impair Allogeneic Engraftment

One hundred percent of recipient (WF) rats conditioned and transplanted with α⊕ and γδ-TCR⁺ T-cell depleted donor marrow engrafted as chimeras (Group C). All of the recipients exhibited stable mixed HSC chimerism with 3.4% to 88.8% of total peripheral lymphoid cells of donor derivation >6 months following BMT. Seventy-five percent and eighty-six percent of recipients transplanted with either αβ-TCR⁺ T-cell (Group A) or γδ-TCR⁺ T-cell (Group B) depleted donor marrow engrafted. The level of donor chimerism in Group A, Group B and Group C was 73.0%±8.3%, 92.3%±9.2% and 46.3%±32.8%, respectively (Table 2).

TABLE 2
PBL typing of mixed allogeneic rat chimeras^{a}

| | | | Bone marrow | | % Donor Chimerism | |
|---|---|---|---|---|---|---|
| | | Depletion of cells | engraftment | | (Mean ± SD) | |
| Group | N | from bone marrow | (n %) | | 30 days | 90 days |
| A | 4 | αβ-TCR | 3 | (75%) | 73 ± | 83.5 ± 6.6 |
| B | 7 | γδ-TCR | 6 | (86%) | 92.3 ± 9.2 | 94.3 ± 3.9 |
| C | 10 | αβ- and γδ-TCR | 10 | (100%) | 46.3 ± 32.8^{b} | 51.1 ± 33.8 |

| | | | | | |
|---|---|---|---|---|---|
| D | 4 | Untreated | NA^{C} | NA | NA |

^{a}Flow cytometric analysis of lymphoid chimerism in PBL from ACI rat to WF rat. Determination of chimerism was performed monthly 1-6 months after bone marrow transplantation. Chimerism was stable in all animals.
^{b}P < 0.05 compared to Group B; P > 0.05 compared to Group A.
^{C}Animals expired between 18 and 28 days after BMT due to severe GVHD.

Control WF rats transplanted with untreated donor ACI rat marrow expired between 18 and 28 days after BMT due to severe GVHD. Survival of recipients of αβ- and γδ-TCW T-cell depleted allogeneic marrow was superior to that for chimeras that received αβ-TCR⁺ or γδ-TCR⁺ T-cell depleted marrow due to avoidance of GVHD in that group.

### Example 3

### Depletion αβ- plus γδ-TCR⁺ T-Cells from Donor Marrow is Required to Prevent GVHD

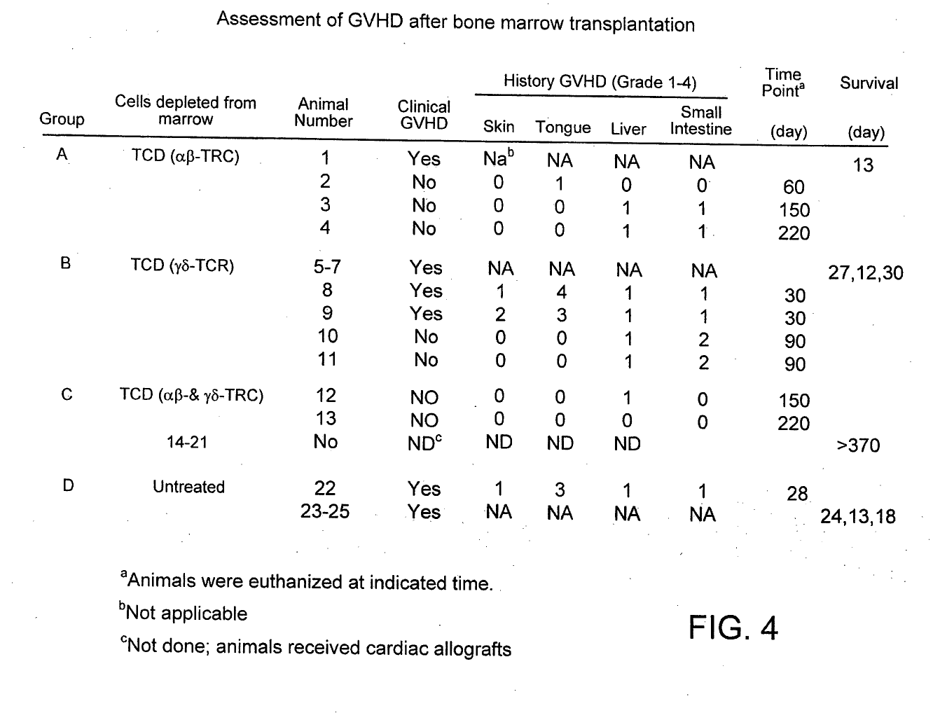

To test whether donor αβ- or γδ-TCR⁺ T-cells would affect the occurrence of GVHD, chimeras were prepared with bone marrow that had been depleted of αβ-TCR⁺ (Group A), γδ-TCR⁺ (Group B), or both αβ- and γδ-TCR⁺ T-cells (Group C). Recipients of untreated marrow were prepared as controls (Group D). In Group D, all four rats conditioned and reconstituted with untreated ACI bone marrow exhibited clinical signs of severe acute GVHD. Three of these animals expired before 28 days due to GVHD. Histologic examination 28 days after BMT in one rat showed severe GVHD consistent with grade 3 in tongue (FIG. 4).

Tissues from animals in Groups A, B and C were collected for histologic assessment of GVHD at 30, 60, 90, 150, and 220 days post BMT. All samples were read blind. The results are summarized in FIG. 4. In Group A, one of the 4 animals exhibited clinical signs of severe GVHD and survived to 13 days post-BMT. After 60 days post-BMT, upon histologic examination of the surviving rats, their tissues displayed mild signs of GVHD consistent with grade 1.

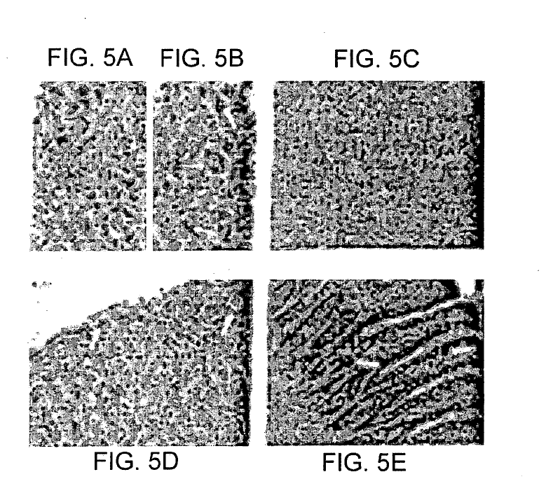

FIGS. 5A-E illustrate histologic assessments of GVHD. Hematoxylin and eosin stained sections of skin, tongue, liver and small intestine were taken from recipient WF rats receiving 100×10⁶ TCD donor marrow depleted of αβ-TCR⁺ T-cells (Group A) or γδ-TCR⁺ T-cells (Group B). Liver sections from a Group A rat 150 days post-BMT showing portal and bile duct inflammation (FIG. 5A, original magnification×150) and apoptosis in different stages of development (FIG. 5B, arrows, original magnification×150). Tongue from a Group B rat 30 days post BMT exhibiting severe inflammation and necrosis of mucosa which is totally denuded. The underlying muscle layer was also inflamed. Granulation tissue with numerous capillaries was also present (FIG. 5C, original magnification×200). The skin from a Group B rat 30 days post-BMT showing moderate mononuclear cell infiltrate in the epidermis as well in dermal layer. Clusters of prominent lymphocytes replace the keratinocytes in the epidermis (arrows). Apoptotic bodies (short arrows) are frequently observed (FIG. 5D, original magnification×150). Small intestine from a Group B rat 90 days post-BMT with evidence of lymphocyte infiltration in the mucosal cells with apoptosis also present (arrows). Regeneration of crypts with mitosis is also noted (FIG. 5E, original magnification×150). The liver revealed mild focal mononuclear cell infiltrate within the portal tracts and in the periductal areas and regenerative change with spotty liver cell necrosis (FIGS. 5A and 5B). Examination of the intestine revealed very mild lymphocytic ileitis with crypt hyperplasia. These data therefore confirm that γδ-TCR⁺ T-cells alone are sufficient to mediate GVHD.

In Group B, 5 of 7 rats exhibited clinical signs of GVHD. Three of the rats died 30 days post-BMT. The remaining two rats showed histological moderate to severe signs of GVHD and necrosis consistent with grade 3 to 4 by 30 days post BMT. The tongue revealed severe inflammation and necrosis (FIG. 5C). The skin revealed moderate mononuclear cell infiltrate in the epidermis which showed rare apoptotic bodies (FIG. 5D). The liver showed mild cholangitis and mild liver cell necrosis. The intestine showed mild lymphocytic ileitis. Ninety days post-BMT, the two rats which showed no clinical signs of GVHD revealed moderate lymphocytic ileitis on histology (FIG. 5E). These data therefore confirm that αβ-TCR⁺ T-cells are the primary effector cells for severe acute GVHD.

None of the animals in which the marrow had been depleted of αβ- and γδ-TCR⁺ T-cells showed clinical signs of GVHD (Group C). However, one rat did have rare lymphocytes within the bile duct epithelium in the liver 150 days post-BMT. One rat displayed no histological evidence of GVHD 220 days post-BMT. These data therefore suggest that both αβ- and γδ-TCR⁺ T-cells mediate clinically significant GVHD, although the severity of GVHD differs. If αβ-TCR⁺ T-cells remain in the marrow inoculum, GVHD is more severe and more frequent compared with γδ-TCR⁺ T-cells.

### Example 4

### Evidence for Tolerance to Donor-Specific Cardiac Allografts

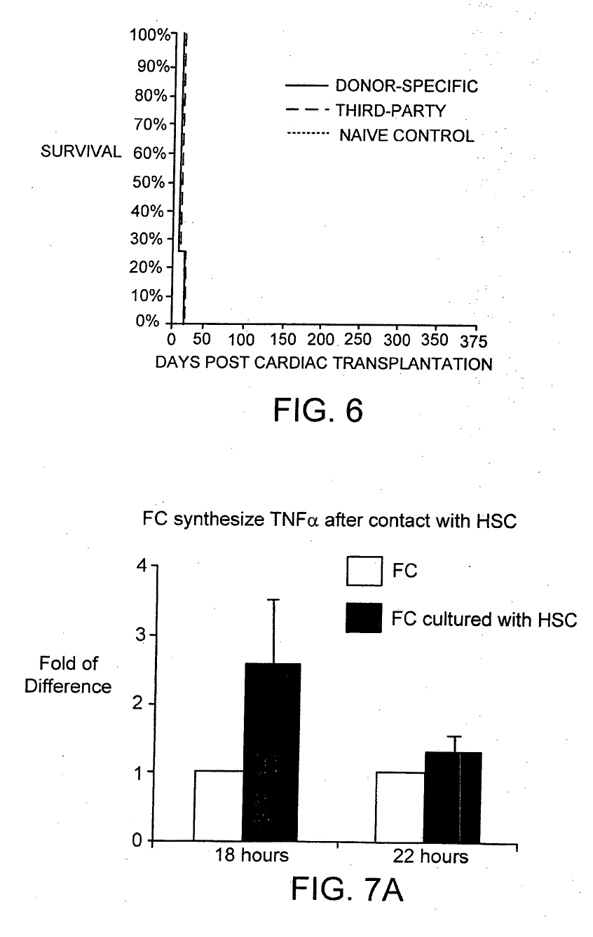

To test whether mixed chimerism achieved with transplantation of marrow depleted of both αβ- and γδ-TCR⁺ T-cells would induce donor-specific tolerance, heterotopic cardiac grafts from ACI (marrow donor) or F344 (third-party) rats were performed. FIG. 6 illustrates the survival of heterotopic cardiac allografts in mixed allogeneic chimeras (ACI→WF). Donor-specific (ACI) or third-party (F344) cardiac grafts were transplanted 4 months after BMT. ACI hearts were transplanted to naive WF rats as controls. Graft survival was determined by palpation and rejection confirmed by pathology. Survival of donor-specific grafts was significantly greater than for third party and controls. As shown in FIG. 6, donor-specific cardiac allografts were permanently accepted by mixed allogeneic chimeras (MST≧375 days), whereas third party (F344) grafts were promptly rejected (MST=15 days). Upon histological examination, all the nonfunctioning grafts had evidence of myocyte necrosis and mononuclear cell infiltration consistent with acute rejection. In contrast, donor-specific allografts showed no evidence for myocytolysis or cellular infiltration. Moreover, there was no evidence for chronic rejection (FIG. 6).

### Example 5

### HSC Stimulate FC to Synthesize TNF-α

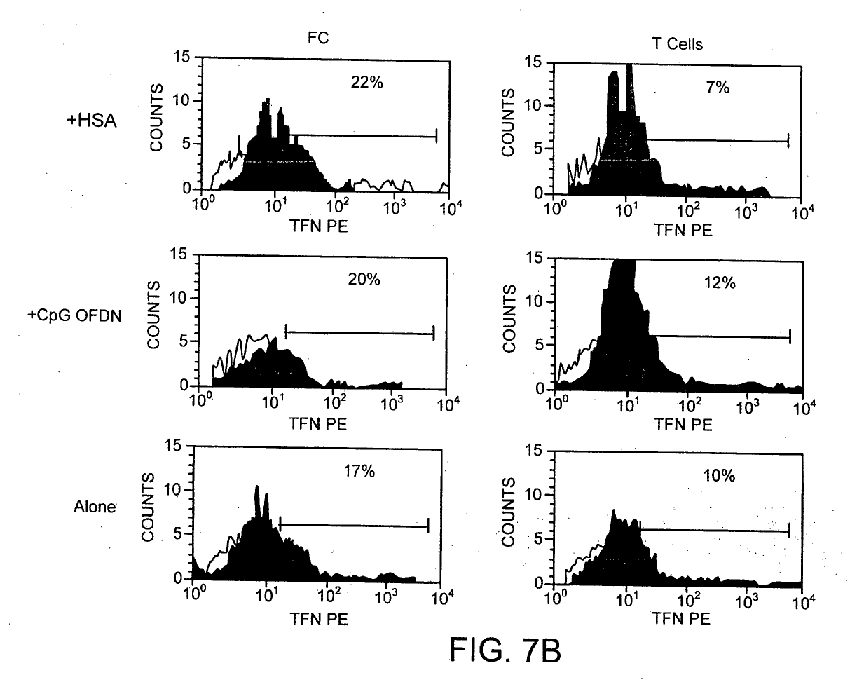

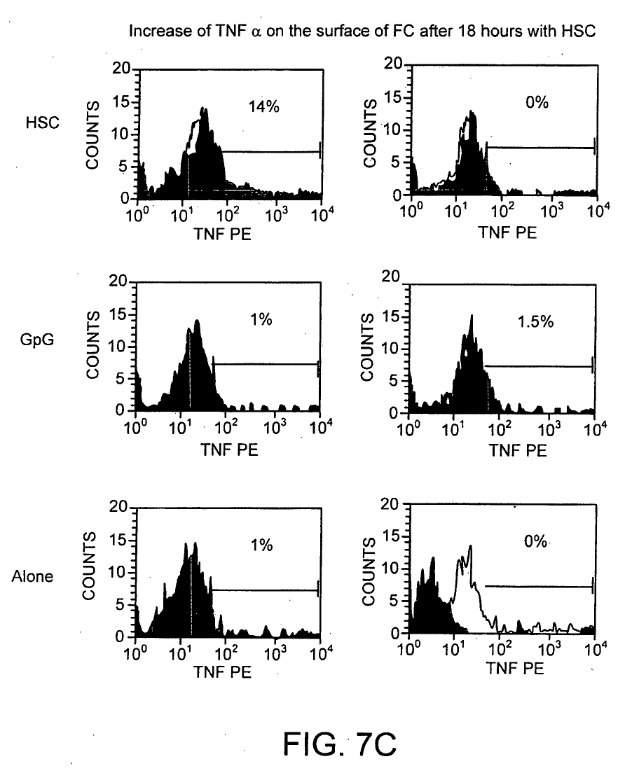

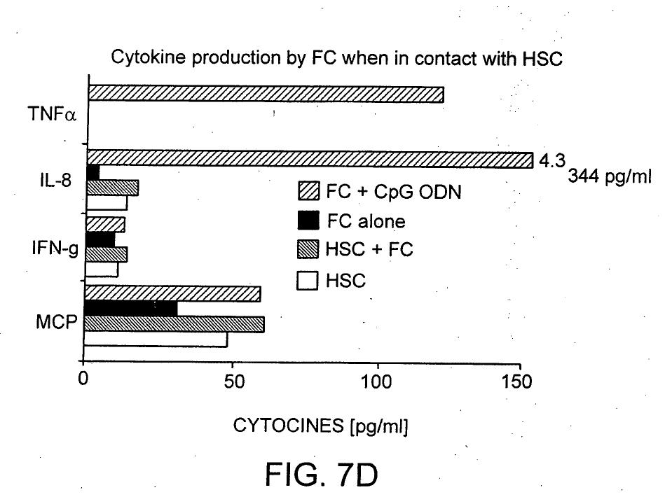

It was previously determined that co-culture of FC with CpG ODN results in production of TNF-α (Fugier-Vivier, et al., J. Exp. Med. 2004; In press). It was therefore evaluated whether co-culture of HSC with FC would similarly result in an increase in production of TNF-α. HSC were co-cultured with FC for 16 and 22 hours. FC were then resorted and mRNA harvested and analyzed by real time RT-PCR for transcript for TNF-α. When FC were co-cultured with HSC for 16 hours, there was a 3-fold increase in TNF-α mRNA compared to FC incubated alone (FIG. 7A). Results are a mean±S.D. of at least 3 experiments. However, this increase was transient, as at 22 hours no difference in the mRNA synthesis was observed.

To confirm production of the protein product for TNF-α with FC, surface and intracellular staining were used for TNF-α in FC after co-culture with HSC. As controls, FC were incubated alone or in the presence of CpG ODN (TLR-9 ligand), shown to activate FC to produce TNF-α (Fugier-Vivier, et al., J. Exp. Med., 2004; In press). After 18 hours of incubation, TNF-α was present in FC intracellularly (FIG. 7B) as well as on the surface (FIGS. 7B and 7C). Only membrane-bound TNF-α was detected when FC were incubated with HSC. Membrane-bound TNF-α was not detectable in FC incubated alone or after CpG ODN stimulation (FIG. 7C). For FIGS. 7B and 7C, data are one representative experiment of 2 and are represented as percent of TNF positive cells in the CD8 positive (FC) population. These data suggest that HSC stimulate FC to produce TNF-α early after contact and that TNF-α is released in the media as well as attached to the membrane of FC.

A number of cytokines play a major role in HSC survival and quiescence. It was therefore determined which cytokines are present in the supernatant of FC+HSC cultures. Sorted HSC were co-cultured with FC at a 1:3 ratio for 18 hours and the cell-free supernatant collected and analyzed by cytokine array (LincoDiagnosis) for production of the following 16 cytokines known to influence hematopoiesis: MIP-1α, GM-CSF, MCP, KC, RANTES, IFN-γ, IL-1β, IL-2, IL-4, IL-5, IL-6, IL-10, IL-12, TNF-α, IL-9, and IL-13. FC or HSC cultured alone or FC cultured in the presence of CpG ODN were used as controls. FC co-cultured with HSC produced MCP, IL-6 and low levels of IFN-γ, but neither TNF-α nor the other cytokines tested were detectable (FIG. 7D). Results are given in pg/mL, for an average of at least 3 samples. Taken together, these data suggest that although TNF-α is produced by FC, the amount released into the media is below the sensitivity of the assay (<3 pb/mL), and may be only a cell-associated form or the TNF-α is being consumed by HSC and/or FC as quickly as it is produced in the cultures.

### Example 6

### Supernatant from FC and HSC Cultured Together Increases Clonogenicity but not Survival of HSC, and is Related to the Presence of TNF-α

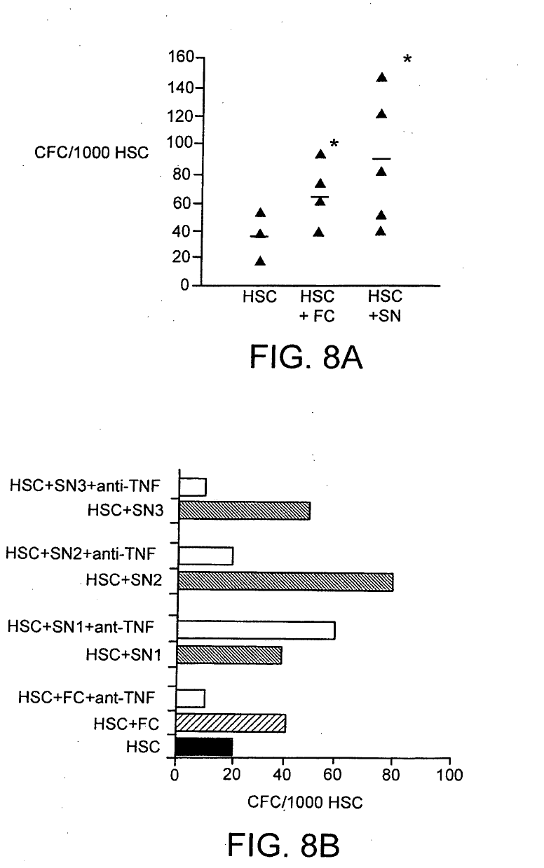

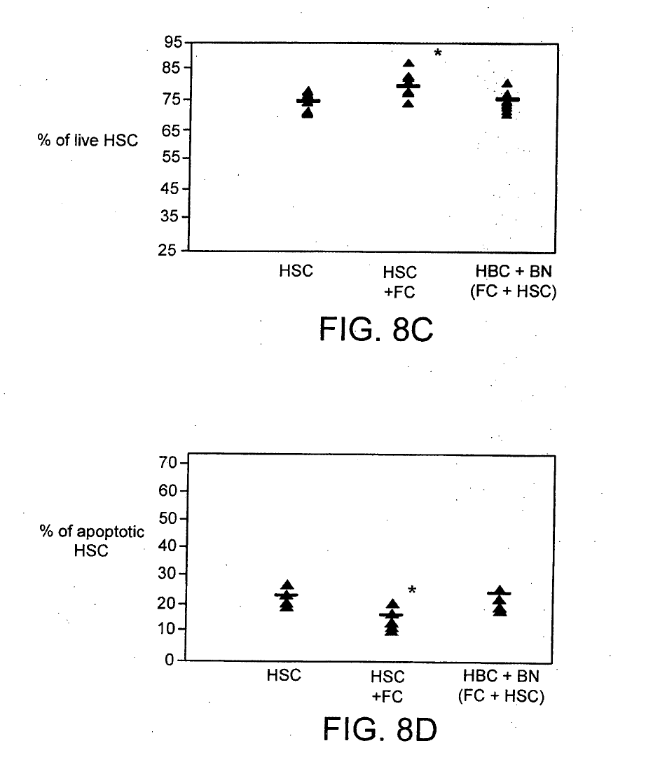

To evaluate whether the cytokines released in the conditioned medium by FC co-cultured with HSC mediate FC activity, sorted KSL cells were preincubated for 18 hours with a 1/10 dilution of supernatants derived from prior 18 hour culture of FC+HSC (FC/HSC SN), then evaluated in a colony-forming cell (CFC) assay as well as apoptosis assays. Controls consisted of FC+HSC or HSC alone. Colonies were evaluated on a reverse microscope. As shown in FIG. 8A, supernatants from FC+HSC culture significantly increased HSC clonogenicity (P<0.01), as well as or even better than FC. In most assays performed, it was found that the FC/HSC SN effect could be reversed by adding anti-TNF-α in the culture before mixing with HSC (FIG. 8B). These data suggest that FC produce TNF-α, and that although a low amount of this TNF-α is detectable in the SN at that concentration, it is sufficient to significantly increase HSC clonogenicity. The fact that this effect on CFC is not always abolished by the addition of anti-TNF-α mAb suggests, however, that other cytokines may be involved in this effect.

It has been previously demonstrated that FC significantly prevent apoptosis of KSL cells in vitro and that this effect is associated with a significant upregulation of Bcl-3 in the HSC. It was therefore examined whether the anti-apoptotic effect of FC on HSC was due to cytokine production. Supernatants were derived from 40 hours culture and then analyzed. FIGS. 8C and 8D show the results of the analyses by flow cytometry using Annexin V and 7AAD labeling. Results are presented as the percentage of live (FIG. 8C) and apoptotic (FIG. 8D) HSC. Data represent 4 to 5 experiments (*significant for P<0.01). The number of CFC in cultures including anti-TNF mAb was compared to those without.

### Example 7

### FC from TNF-α^{-/-} Mice do not Facilitate HSC Engraftment

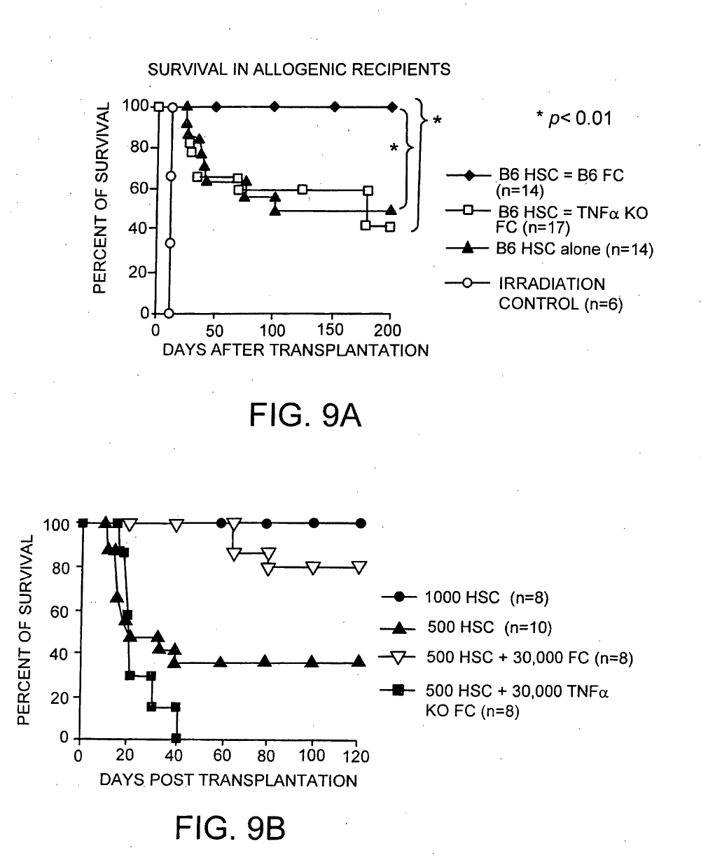

FC significantly enhance engraftment of allogeneic HSC and limiting numbers of syngeneic HSC (Kaufman C L, et al., Blood 1994;84:2436-2446; Huang Y, et al., Blood 2004;104:873-880; Grimes H L, et al., Experimental Hematology 2004, In press). To further confirm a direct biologic role for TNF-α in FC function, TNF-α-deficient mice (TNF-α^{-/-}) (B6, H-2^{b} background) were used as FC donors. The effect of wild type (WT) FC and TNF-α^{-/-} FC were compared on WT HSC (B6) engraftment first using the allogeneic model: B6 into B10.BR (H-2^{k}). FC from TNF-α^{-/-} mice were not functional as there was no significant enhancement in engraftment compared to HSC alone (41% recipient survival versus 50%, respectively), and facilitation was significantly impaired (P<0.991) compared to wild type FC+HSC. In contrast, all recipients of HSC and WT FC engrafted durably (FIG. 9A).

In addition, the present teachings show that the function of FC from TNF-α^{-/-} donors was significantly impaired compared to wild type FC when HSC numbers are limiting in syngeneic recipients. In the syngeneic setting, while 100 B6 HSC rescue 100% of the recipients, 500 HSC rescue only 30% of syngeneic recipients (Grimes H L, et al., Experimental Hematology 2004, In press). The addition of 30,000 FC results in significantly enhanced engraftment. In this syngeneic model, FC from TNF-α^{-/-} mice did not improve engraftment of 500 HSC, as none of the recipients survived more than 40 days after transplantation (FIG. 9B). These data show a significant impairment of the facilitating activity of FC from TNF-α^{-/-} mice on enhancement of HSC engraftment.

### Example 8

### FC from TNF-α^{-/-} Mice do not Affect HSC Clonogenicity

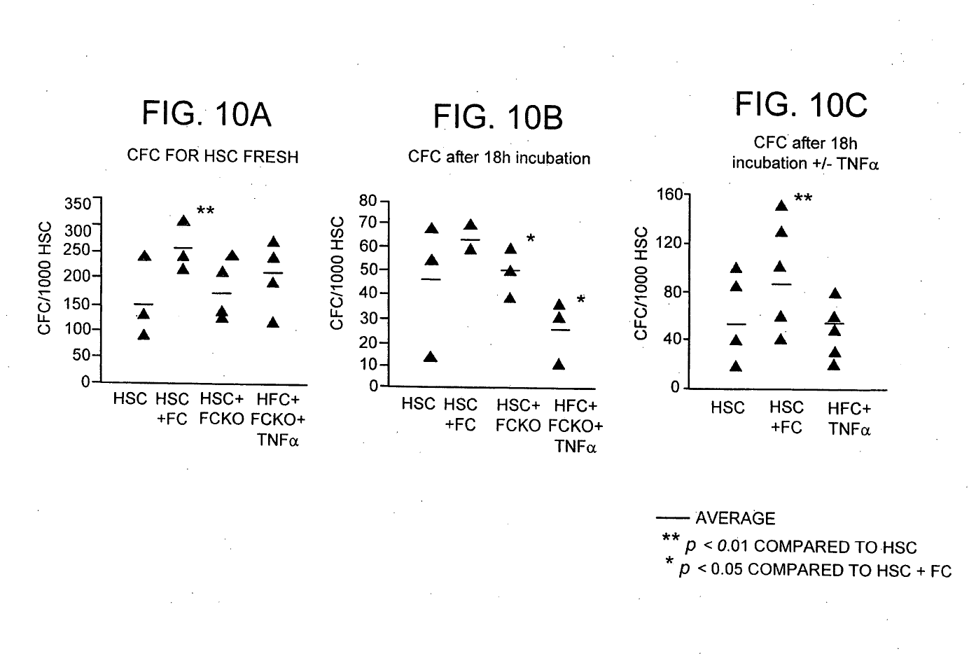

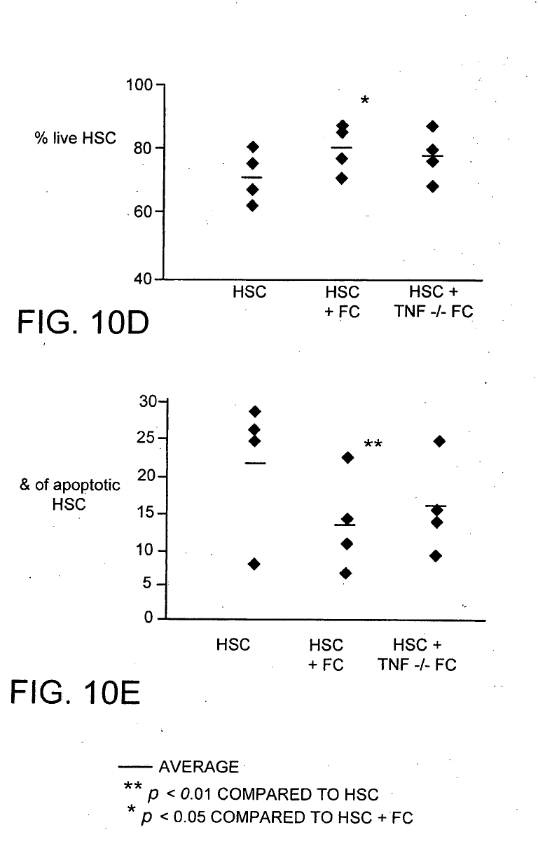

It was previously demonstrated that FC significantly increase HSC clonogenicity in vitro. Using the colony forming cells (CFC) assay, the ability of FC from TNF-α^{-/-} mice to affect WT HSC differentiation in vitro was evaluated. Sorted FC from TNF-α^{-/-} or from WT (B6) mice were mixed with B6 HSC (3:1 ratio) and cultured in methylcellulose containing growth factors (as described previously) for 14 days. Colonies were evaluated on an inverted microscope; results are given as number of CFC for 1,000 HSC, data represent at least 3 different experiments (* significant for P<0.01 and ** for P<0.05). FC from TNF-α^{-/-} mice did not improve HSC clonogenicity and the number of colonies was significantly lower than when HSC were co-cultured with wild type FC (FIG. 10A).

Notably, preincubation of TNF-α^{-/-} FC with TNF-α (10 ng/mL) for one hour before culture with HSC only slightly restored the effect of FC on HSC (FIG. 10A). The inventor also assessed FC from TNF-α^{-/-} mice in the CFC assay. In these experiments, TNF-α^{-/-} were FC co-cultured with HSC for 18 hours. FC from TNF-α^{-/-} donors did not affect HSC clonogenicity and were significantly impaired when they had been pretreated with TNF-α (25.0+13.2 CFC for 1000 HSC (FIG. 10B). The TNF-α dose (10 ng/mL) used on TNF-α^{-/-} FC did not stimulate HSC clonogenicity by itself (FIG. 10C) as previously reported (Jacobsen F W, et al., J. Immunol. 1995;154:3732-3741). These data indicate that FC from TNF-α^{-/-} donors are impaired in function. The transient effect of TNF-α^{-/-} FC treated with TNF-α could be due to the attachment (or fixation) of the TNF-cl on the surface of FC that is no longer present 18 hours later.

### Example 9

### FC from TNF-α^{-/-} Mice are Impaired in Their Ability to Increase HSC Survival

It was recently reported that co-culture of FC with HSC significantly increases survival of HSC in vitro via prevention of apoptosis. In the present studies, it was explored whether the anti-apoptotic effect of FC is due to TNF-α secretion by FC. FC from TNF-α^{-/-} mice were compared to WT FC in their ability to increase HSC survival after incubation, using Annexin V and 7AAD labeling to determine the proportion of live cells versus apoptotic or dead cells. The same gating strategy described previously was used: Sorted HSC were incubated at 1:3 ratio with or without WT FC or TNF-α^{-/-} FC (FC KO) for 40 hours and analyzed for live (FIG. 10D) versus apoptotic (FIG. 10E) HSC. After incubation, cells were stained with C-kit APC, Annexin V and 7AAD, and cells in the lymphoid gate and c-kit⁺ (HSC) or c-kit⁻ (FC) were analyzed for Annexin V and 7AAD expression. Debris was excluded and cells in the lymphoid gate gated for ck-kit+ cells and Annexin V versus 7AAD labeling. Annexin V− 7AAD− cells are alive, Annexin V+ 7AAD− are apoptotic, and Annexin V+ 7AAD+ are dead. Data represent 4 experiments in duplicate (* significant for P<0.01, ** significant for P<0.05). Notably, TNF-α^{-/-} FC did not significantly increase HSC survival, while the WT FC did (FIG. 10D). These data strongly support a role for TNF-α produced by FC on HSC function and/or survival.

### Example 10

### Blocking TNF-α in Culture Inhibits the FC Effect on HSC Clonogenicity

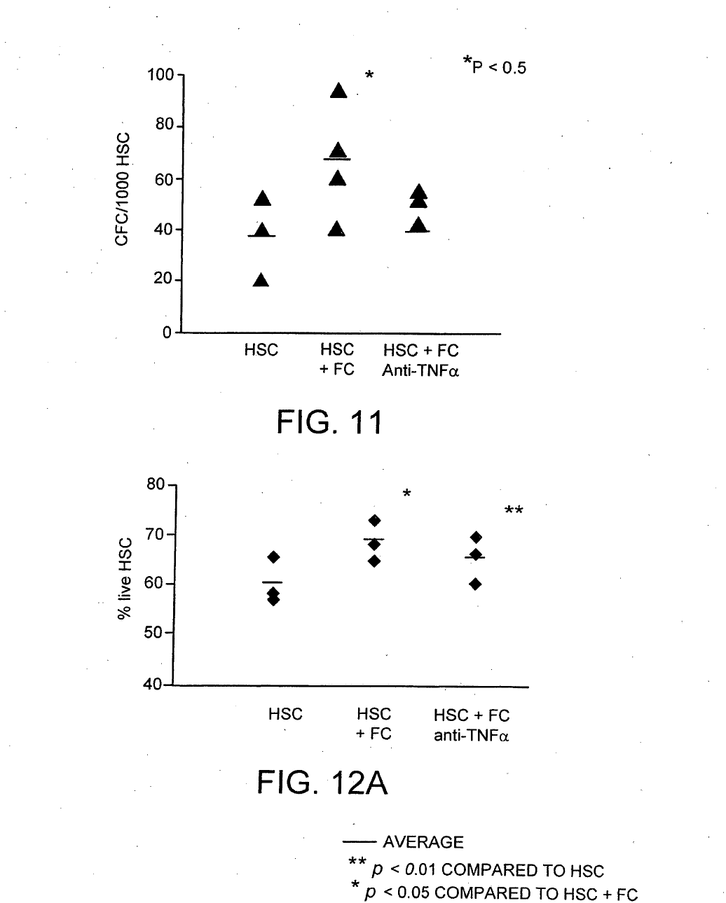

To further confirm the role played by TNF-α in FC function, neutralizing anti-TNF-α antibodies were used to block TNF-α on WT assessed outcome on HSC in the CFC assay. HSC were preincubated for 18 hours alone or at 1:3 ratio (HSC:FC) in the presence of FC, or FC pretreated with anti-TNF-α neutralizing antibody for 1 hour, and then evaluated for CFC after culture in methylcellulose-containing growth factors for 14 days. Results, shown in FIG. 11, are given as number of CFC for 1,000 HSC, data represent at least 4 different experiments (* significant for P<0.01 and ** significant for P<0.05). The increase of clonogenicity obtained when HSC are incubated in the presence of FC was completely abolished when TNF-α was neutralized in the culture (FIG. 11). These data show that FC lose their effect on HSC clonogenicity when TNF-α is neutralized and confirm a major role for TNF-α in FC function.

### Example 11

### Blocking TNF-α in the Culture Inhibits the Ability of FC to Increase HSC Survival and Bcl-3 Transcription in the HSC

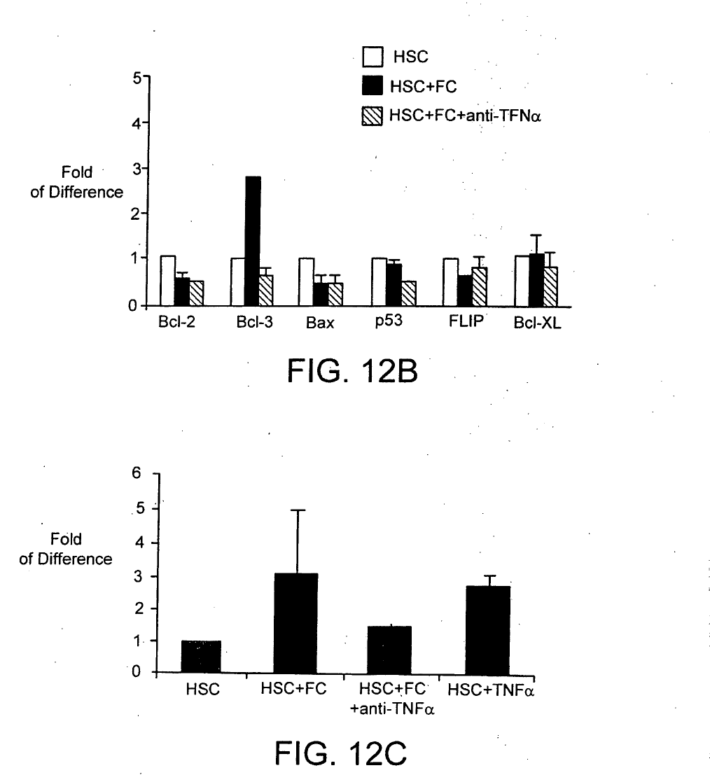

It was previously reported that FC enhance survival of HSC in vitro and in vivo by preventing apoptosis. To determine the mechanism underlying this effect, HSC were preincubated for 40 hours alone or at 1:3 ratio (HSC:FC) in the presence of FC, or FC pretreated with anti-TNF-α neutralizing antibody for 1 hour and then evaluated by flow cytometry for survival using Annexin V and 7AAD labeling. Data (FIG. 12A) represent 3 to 4 experiments in duplicate (* significant for P<0.01). As observed previously, the percentage of live cells was significantly increased when HSC were incubated with FC (59.9±4.4% for HSC alone versus 68.0±4.1% for HSC plus FC; P=0.008) (FIG. 12A). However, when FC were preincubated with anti-TNF-α before co-culture with HSC, HSC survival was significantly lower than when co-cultured with untreated FC (64.3±4.7% HSC survival, P=0.03).

Since the results described herein demonstrate that the anti-apoptotic effect of FC on HSC was associated with a significant increase in the Bcl-3 transcription in the HSC, the effect of neutralizing anti-TNF-α antibodies on the upregulation of Bcl-3 in HSC was evaluated. HSC were analyzed for the presence of the anti-apoptotic regulatory proteins Bcl-2, Bcl-x_{L}, Bcl-3, FLIP, p53 or proapoptotic Bax and p53, using real time RT-PCR, when HSC were incubated for 16 hours in the presence of FC or FC preincubated with anti-TNF-α mAb in the same conditions as for the apoptosis study. As expected, the mRNA level of Bcl-3 was increased when the HSC incubated with FC (FIG. 12B). However when TNF-α was blocked with anti-TNF-α, the transcription for Bcl-3 was significantly reduced. These data show that TNF-α is involved in the ability of FC to upregulate Bcl-3 transcription in HSC. Moreover, when HSC were incubated for 16 hours with TNF-α (10 ng/mL), Bcl-3 transcription was similarly increased, as with FC (FIG. 12C) which confirmed further the role of TNF-α in the modulation of Bcl-3 transcription in the HSC.

### Example 12

### FC from Wild Type Donors Partially Restore Engraftment of TNF-α Receptor Deficient HSC

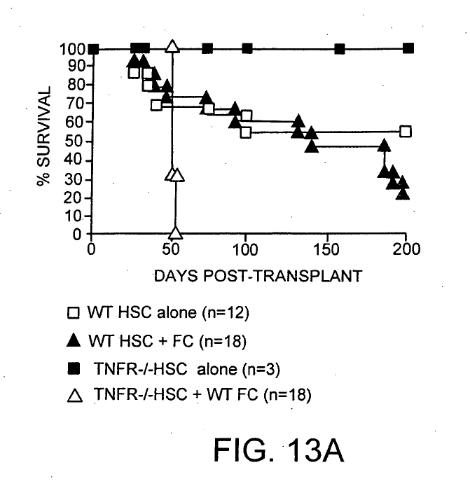

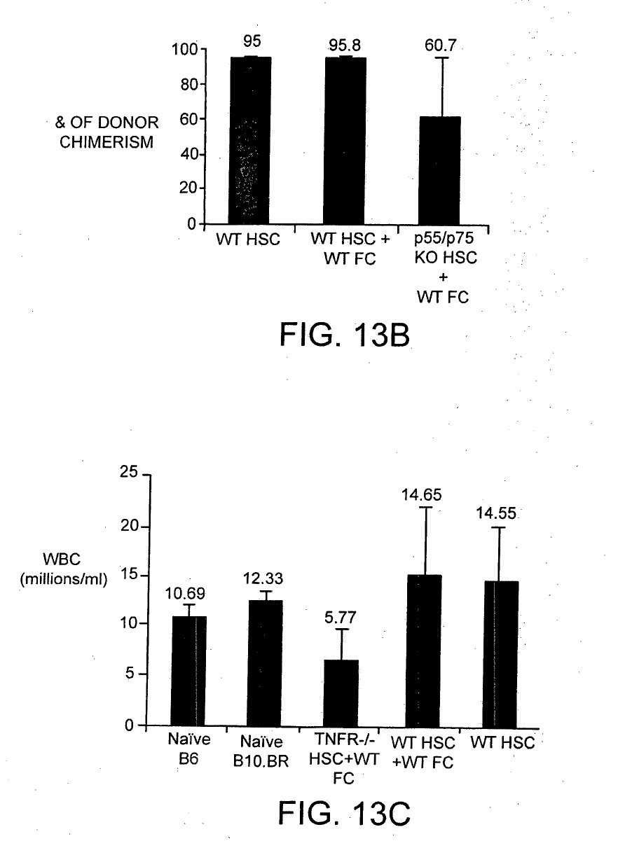

To further evaluate the role played by TNF-α in FC activity in vivo, the effect of wild-type (WT) FC on the engraftment of HSC from TNF-α p55p75 receptor deficient (TNFR^{-/-}) mice (B6 background, H-2^{b}) was evaluated in allogeneic recipients. HSC from TNFR^{-/-} mice showed less engraftment efficiency in allogeneic recipients (B10.BR) than the WT HSC. WT FC were able to facilitate TNFR^{-/-} HSC short-term engraftment in B10.BR recipients (from 0% recipient survival for TNFR^{-/-} HSC alone to 47% recipient survival for TNFR^{-/-} HSC plus WT FC at 180 days). FIG. 13A shows a survival curve for B10.BR mice transplanted with allogeneic B6 HSC. B10.BR mice were conditioned with 950 cGy TBI and transplanted with 10,000 B6 HSC (□) in the presence or absence of 30,000 FC from B6 (▪) or with 10,000 HSC from TNFR^{-/-} mice (Δ) in presence of in absence of 30,000 WT FC (▴). Data represent 3 different experiments.

However, the survival of recipients of TNFR^{-/-} HSC plus FC deceased rapidly thereafter, and was only 20% at 200 days. The analysis of donor repopulation at 3 months in peripheral blood of recipients B10.BR (FIG. 13B) show a lower percentage of donor chimerism in recipients of TNFR^{-/-} HSC+FC compared to WT HSC+FC (not significant) and the white blood cell count (FIG. 13C) is much lower in recipients of TNFR/− HSC+WT FC, confirming that the engraftment is not total. These data suggest that the effect of FC on HSC engraftment in allogeneic recipients is not entirely related to the presence of TNF-α. Nevertheless, when HSC from TNFR^{-/-} mice were assayed for CFC, they generated fewer colonies than WT HSC in methylcellulose, and there was no effect of WT FC on TNFR^{-/-} HSC clonogenicity (data not shown). Taken together, these data suggest that the presence of TNF-α receptors on HSC is not critical for the facilitating effect in vivo to occur, but is necessary for the increase of the clonogenicity and long-term engraftment.

The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof.

The foregoing description is considered as illustrative only of the principles of the invention. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims which follow.

What is claimed is:

1. A method of increasing HSC engraftment survival, comprising:
co-incubating prior to engraftment a pharmaceutical composition that stimulates TNF-α expression and a cellular composition comprising human hematopoietic stem cells to form a mixture, wherein said stem cells are depleted of graft-versus-host-disease-producing cells having a phenotype of αβTCR+ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and
administering said mixture to said mammal.

2. A method of increasing HSC engraftment survival in a mammal, comprising:
co-incubating prior to engraftment a pharmaceutical composition that stimulates upregulation of Bcl-3 by said HSC and a cellular composition comprising human hematopoietic stem cells to form a mixture, wherein said stem cells are depleted of graft-versus-host-disease-producing cells having a phenotype of αβTCR+ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and
administering said mixture to said mammal.

3. A method of increasing HSC engraftment survival in a mammal, comprising:
administering to said mammal a cellular composition comprising human hematopoietic stem cells, wherein said stem cells are depleted of graft-versus-host-disease-producing cells having a phenotype of αβTCR+ with the retention of mammalian hematopoietic facilitatory cells having a phenotype of CD8+/TCR+, CD8+/TCR−, which hematopoietic facilitatory cells are capable of facilitating engraftment of bone marrow cells; and
concurrently therewith or subsequent thereto, administering to said mammal a pharmaceutical composition that stimulates TNF-α, expression.

4. A method of increasing HSC engraftment survival in a mammal, comprising:
administering to said mammal a cellular composition comprising human hematopoietic stem cells, wherein said stem cells are depleted of graft-versus-host-disease-producing cells having a phenotype of αβTCR+ with the retention of mammalian hematopoietic facilitatory cells having a phenotype of CD8+/TCR+, CD8+/TCR−, which hematopoietic facilitatory cells are capable of facilitating engraftment of bone marrow cells; and
concurrently therewith or subsequent thereto, administering to said mammal a pharmaceutical composition that increases upregulation of Bcl-3 by said HSC.

5. A cellular composition comprising:
a) mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and
b) a pharmaceutical composition that provides an increased amount of TNF-α.

6. The composition of claim 5, wherein said pharmaceutical composition comprises TNF-α.

7. The composition of claim 5, wherein said pharmaceutical composition comprises an agent that stimulates expression of TNF-α from said facilitatory cells.

8. A cellular composition comprising:
a) mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and
b) a pharmaceutical composition that provides an increased amount of Bcl-3.

9. The composition of claim 8, wherein said pharmaceutical composition comprises Bcl-3.

10. The composition of claim 8, wherein said pharmaceutical composition comprises an agent that increases expression of Bcl-3.

11. A method of partially or completely reconstituting a mammal's lymphohematopoietic system comprising:
(a) administering to the mammal a cellular composition comprising mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and
(b) stimulating the expression of TNF-α from said facilitatory cells and/or increasing the ability of said facilitatory cells to upregulate Bcl-3 in said hematopoietic stem cells.

12. The method of claim 11, wherein said stimulation comprises introducing to said facilitatory cells a pharmaceutical composition that increases TNF-α expression prior to administration to said mammal.

13. The method of claim 11, wherein said increase in upregulation is effected by introducing to said facilitatory cells a pharmaceutical composition that increases said upregulation of Bcl-3 prior to said administration.

14. The method of claim 11, comprising administering to said mammal a pharmaceutical composition that increases TNF-α expression subsequent to said administration of said cellular composition.

15. The method of claim 11, comprising administering to said mammal a pharmaceutical composition that increases said upregulation of Bcl-3 subsequent to said administration of said cellular composition.

16. A method of inducing tissue or organ regeneration in a mammal comprising:
(a) administering to the mammal a cellular composition comprising mammalian hematopoietic stem cells, wherein said stem cells are depleted of graft-versus-host-disease-producing cells having a phenotype of αβTCR⁺ with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells; and
(b) stimulating the expression of TNF-α from said facilitatory cells and/or increasing the ability of said facilitatory cells to upregulate Bcl-3 in said hematopoietic stem cells.

17. The method of claim 16, wherein said stimulation comprises introducing to said facilitatory cells a pharmaceutical composition that increases TNF-α expression prior to administration to said mammal.

18. The method of claim 16, wherein said increase in upregulation is effected by introducing to said facilitatory cells a pharmaceutical composition that increases said upregulation of Bcl-3 prior to said administration.

19. The method of claim 16, comprising administering to said mammal a pharmaceutical composition that increases TNF-α expression subsequent to said administration of said cellular composition.

20. The method of claim 16, comprising administering to said mammal a pharmaceutical composition that increases said upregulation of Bcl-3 subsequent to said administration of said cellular composition.

21. A method of protecting mammalian hematopoietic stem cells from apoptosis, comprising:
depleting said stem cells of graft-versus-host-disease-producing cells having a phenotype of αβTCR⁺ while retaining mammalian hematopoietic facilitatory cells having a phenotype of CD8⁺/TCR⁺, CD8⁺/TCR⁻, which hematopoietic facilitatory cells are capable of facilitating engraftment of bone marrow cells; and
contacting said depleted stem cells with a pharmaceutical composition that increases the amount of TNF-α.

22. The method of claim 21, wherein said pharmaceutical composition comprises TNF-α.

23. The method of claim 21, wherein said pharmaceutical composition comprises an agent that increases expression of TNF-α from said facilitatory cells.

24. A method of protecting mammalian hematopoietic stem cells from apoptosis, comprising:
depleting said stem cells of graft-versus-host-disease-producing cells having a phenotype of αβTCR⁺ while retaining mammalian hematopoietic facilitatory cells having a phenotype of CD8⁺/TCR⁺, CD8⁺/TCR⁻, which hematopoietic facilitatory cells are capable of facilitating engraftment of bone marrow cells; and
contacting said depleted stem cells with a pharmaceutical composition that increases upregulation of Bcl-3.
